(19) United States
(12) Patent Application Publication
(10) Pub. No.: US 2017/0096636 A1
(43) Pub. Date: Apr. 6, 2017

(54) METHODS AND COMPOUNDS FOR PHOTOTHERAPY WITH CHALCOGENORHODAMINE PHOTOSENSITIZERS

(71) Applicants: Wake Forest University Health Sciences, Winston-Salem, NC (US); The Research Foundation for the State University of New York, Amherst, NY (US)

(72) Inventors: Zachariah A. McIver, Winston-Salem, NC (US); Jacqueline E. Hill, Buffalo, NY (US); Michael R. Detty, Rochester, NY (US)

(21) Appl. No.: 15/316,016

(22) PCT Filed: Jun. 4, 2015
(86) PCT No.: PCT/US15/34187

§ 371 Date: Dec. 2, 2016

Related U.S. Application Data
(60) Provisional application No. 62/008,161, filed on Jun. 5, 2014.

(51) Int. Cl. C12N 5/00; A61K 31/4745; A61K 31/4743; A61K 35/26; A61K 41/00

(52) U.S. Cl. CPC C12N 5/0087; A61K 35/26; A61K 41/0057; A61K 31/4743; A61K 31/4745

(57) ABSTRACT

A method of selectively depleting pathogenic T lymphocytes from a blood cell composition is carried out by (a) combining the cell composition ex vivo with an active compound in an effective amount, and then (b) irradiating the cells with light ex vivo for a time and at an intensity sufficient to selectively kill pathogenic T lymphocytes in said cell composition. Chalcogenorhodamine photosensitizers useful as such active compounds are also described.

### FIELD OF THE INVENTION

The present invention concerns phototherapy methods for use in selectively depleting pathogenic T lymphocytes from a blood cell composition ex vivo and compounds useful therein.

### BACKGROUND OF THE INVENTION

Extracorporeal photopheresis (ECP) has been used successfully for more than 30 years in the treatment of erythrodermic cutaneous T cell lymphoma (CTCL), and more recently has shown promising results in several T cell mediated disorders, including systemic sclerosis, treatment and prevention of solid organ rejection, graft-versus-host disease, and Crohn's disease.¹ Although response rates vary depending on disease and disease status, the use of ECP may facilitate control of disease and improve overall survival. However, not all patients obtain a significant or durable response,² indicating that improvements in the procedure warrant investigation.

During ECP, lymphocytes are collected and exposed to 8-methoxypsoralen (8-MOP) and are then irradiated with UVA (PUVA), which cross-links DNA within the nuclei of the cells and induces apoptosis. The subsequent reinfusion of the apoptotic lymphocytes produces an immunomodulatory effect. Although the mechanism of ECP is not well established, a vaccination effect is hypothesized to occur against malignant and alloreactive cells. After reinfusion of apoptotic lymphocytes, phagocytosis by antigen-presenting cells (APCs) of membrane markers of alloreactive and malignant T cells induces cytotoxic T cell (CTL) responses. Disease control is then mediated through CTLs with disease specificity.³

However, 8-MOP is a non-selective photosensitizer, which may in part contribute to its limited efficacy. The fact that DNA cross-linking by 8-MOP is indiscriminate and occurs in all cells results in non-malignant and resting lymphocytes significantly contributing to the apoptotic milieu. Reinfusion of these non-targeted cells may serve to limit the production of disease specific CTLs by competitively reducing the presentation of disease specific antigens, or by the induction of tolerance to prominent lymphocyte antigens.^{4,5} Consequently, the efficiency of ECP may be improved with the use of a selective photosensitizer.

Accordingly, there is a need for more selective photosensitizers for use in ECP and related procedures.

### SUMMARY OF THE INVENTION

A first aspect of the present invention is a method of selectively depleting pathogenic T lymphocytes from a blood cell composition. The method comprises: (a) combining the cell composition ex vivo with an active compound as described herein in an effective amount thereof, and then (b) irradiating the cell composition with light ex vivo for a time and at an intensity sufficient to selectively kill pathogenic T lymphocytes in the cell composition.

A further aspect of the present invention is active compounds as described herein, e.g., for use in carrying out a method as described above, and further described below.

The present invention is explained in greater detail in the drawings herein and the specification set forth below. The disclosures of all United States patent references cited herein are to be incorporated by reference herein in their entirety.

### BRIEF DESCRIPTION OF THE DRAWINGS

FIG. 1. Decays of phosphorescence from ¹O₂ sensitized by selenorhodamines 1-Se—Cl-6-Se—Cl. The signal obtained from air-saturated MeOH in the cuvette was used as the instrument response function (IRF).

FIG. 2. Flow cytometric analysis of photosensitizer retention in activated versus resting T cells. Chalcogenorhodamine photosensitizers are preferentially retained in stimulated T cells. A) Stimulated T cells by were identified by FACS analysis for CD3+ and CD25+ coexpression. Resting T cells were identified by CD3+ without expression of CD25. B) The ratio of the mean fluorescent intensity (MFI) of stimulated versus resting T cells for all photosensitizer was determined, and represented by bar graphs for the amide/thioamide and thiorhodamine/selenorhodamine scaffolds. Mean±SE are plotted. * p<0.05, ** p<0.01.

FIG. 3. The effects of intracellular resident times on dark toxicity. (A) The effects of PD on the bioenergetics of resting T cells using 2-Se—Cl and 2-S—Cl is compared to control in a basal state, and after the addition of oligomycin (to block ATP synthesis), FCCP (to uncouple ATP synthesis from the electron transport chain), and rotenone (to block complex I of the electron transport chain) for one representative experiment. (B) Bar graphs represent mean oxygen consumption rate (OCR) of 12 photosensitizers as a percent of control in resting T cells for (A) the amide- and thioamide-containing analogues, and for (B) the julolidine and half-julolidine scaffolds.

FIG. 4. Phototoxicity of chalcogenorhodamine photosensitizers. Bar graphs demonstrate the effects of PD on the OCR and survival of cells compared to control for photosensitizers 2-S—Cl, 2-Se—Cl, 4-S—Cl, and 4-Se—Cl. Three donors were used in 4 independent experiments. Mean±SE are plotted. * p<0.05 compared to control.

FIG. 5. The effects of photodepletion with 2-Se—Cl on the bioenergetics and survival of activated T cells. PBMCs were stimulated with 50 ng/mL staphylococcal enterotoxin B (SEB) for 72 hours and then photodepleted (PD) with 5×10⁻⁸ M of 2-Se—Cl and 5 J/cm² light. (A) The bar graphs represent the average area under the curve (AUC) summations for basal OCR/baseline OCR and (B) the ECAR measurements for resting and activated T cells of PD and non-PD (control) samples. (C) Cell survival was measured 18 hours after light exposure and enumerated by FACS analysis by exclusion of Annexin V and 7AAD. D) Percent survival compared to control was determined in 3 independent experiments. Mean±SE are plotted. ** p<0.01.

FIG. 6. Photodepletion with 2-Se—Cl selectively depletes immune responses. (A) PBMCs were stimulated with 50 ng/mL staphylococcal enterotoxin B (SEB) for 72 hours, and then photodepleted (PD) with 5×10⁻⁸ M of 2-Se—Cl and 5 J/cm² light. Cells were then rested overnight, stained with CFSE, and rechallenged with SEB or toxic shock syndrome toxin 1 (TSST-1) in culture for 6 days. Histograms of CFSE fluorescence for stimulated (dashed lines) and non-stimulated (solid lines) T cells are shown for one representative sample. (B) Bar graph represents the percent of the total cells proliferating in response to SEB or TSST-1 for PD and control (non-PD) samples. C) Bar graph represents the division index (average # of cell divisions for all cells) and proliferation index (the average # of divisions for proliferating cells) for TSST-1 stimulated cells. Non-PD cells were used as control. Three donors were used in 3 independent experiments. Mean±SE are plotted. ** p<0.01.

### DETAILED DESCRIPTION OF ILLUSTRATIVE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

### 1. Definitions.

“Subject” or “patient” as used herein (and including both “donors” and “recipients” where different) are in general, mammalian subjects, including both human subjects and other mammalian subjects (e.g., dog, cat, horse, etc.) for veterinary purposes. Subjects may be male or female and may be of any suitable age, including neonate, infant, juvenile, adolescent, adult, and geriatric subjects.

“Anion” as used herein includes, but is not limited to, halides, sulfonates, carboxylates, hexafluorophosphate, and tetrafluoroborate. In some preferred embodiments, the anion is tosylate, acetate, or chloride, particularly chloride.

“Alkyl” as used herein alone or as part of another group, refers to a straight or branched chain hydrocarbon containing from 1 to 6, 8 or 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Loweralkyl” as used herein, is a subset of alkyl, in some embodiments preferred, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. Alkyl and loweralkyl groups can be unsubstituted or substituted with one or more (e.g., one, two, three four, etc.) independently selected electron-donating or electron-withdrawing groups.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be substituted with one or more (e.g., one, two, three four, etc.) independently selected electron-donating or electron-withdrawing groups.

“Heteroaryl” as used herein refers to a monovalent aromatic group having a single ring or two fused rings and containing in the ring(s) at least one heteroatom (typically 1 to 3) selected from nitrogen, oxygen or sulfur. Unless otherwise defined, such heteroaryl groups typically contain from 5 to 10 total ring. Representative heteroaryl groups include, by way of example, monovalent species of pyrrole, imidazole, thiazole, oxazole, furan, thiophene, triazole, pyrazole, isoxazole, isothiazole, pyridine, pyrazine, pyridazine, pyrimidine, triazine, indole, benzofuran, benzothiophene, benzoimidazole, benzthiazole, quinoline, isoquinoline, quinazoline, quinoxaline and the like, where the point of attachment is at any available carbon or nitrogen ring atom. The term “heteroaryl” is intended to include both substituted and unsubstituted heteroaryl unless otherwise indicated and these groups may be substituted with one or more (e.g., one, two, three four, etc.) independently selected electron-donating or electron-withdrawing groups.

“Electron-withdrawing” and “electron donating” refer to the ability of a substituent to withdraw or donate electrons relative to that of hydrogen if the hydrogen atom occupied the same position in the molecule. These terms are well understood by one skilled in the art and are discussed in Advanced Organic Chemistry, by J. March, John Wiley and Sons, New York, N.Y., pp. 16-18 (1985), incorporated herein by reference. Examples of such electron withdrawing and electron donating groups or substituents include, but are not limited to halo, nitro, cyano, carboxy, alkylcarboxy, loweralkenyl, loweralkynyl, loweralkanoyl (e.g., formyl), carboxyamido, aryl, quaternary ammonium, aryl (loweralkanoyl), carbalkoxy and the like; acyl, carboxy, alkanoyloxy, aryloxy, alkoxysulfonyl, aryloxysulfonyl, and the like; hydroxy, alkoxy or loweralkoxy (including methoxy, ethoxy and the like); loweralkyl; amino, alkylamino, lower alkylamino, di(loweralkyl) amino, aryloxy (such as phenoxy), mercapto, loweralkylthio, lower alkylmercapto, disulfide (loweralkyldithio) and the like; 1-piperidino, 1-piperazino, 1-pyrrolidino, acylamino, hydroxyl, thiolo, alkylthio, arylthio, aryloxy, alkyl, ester groups (e.g., alkylcarboxy, arylcarboxy, heterocyclocarboxy), azido, isothiocyanato, isocyanato, thiocyanato, cyanato, and the like. One skilled in the art will appreciate that the aforesaid substituents may have electron donating or electron withdrawing properties under different chemical conditions. Moreover, the present invention contemplates any combination of substituents selected from the above-identified groups. See U.S. Pat. Nos. 6,133,261 and 5,654,301; see also U.S. Pat. No. 4,711,532.

### 2. Active Compounds.

Active compounds for use in the present invention include compounds of Formula I:

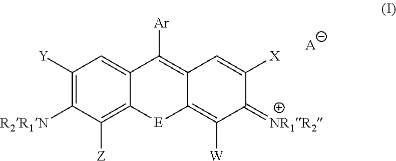

wherein:

E is S or Se;

Ar is aryl (e.g., phenyl) or heteroaryl (e.g., 2-thienyl), each of which is substituted or unsubstituted;

W, X, Y, and Z are each independently H or C1 through C8, linear or branched, alkyl;

R₁′, R₂′, R₁″ and R₂″ are each independently H or C1 through C8, linear or branched, alkyl; and/or

R₁′ and R₂′ are alkyl groups connected such that they together comprises a 3, 4, 5, 6 or 7-membered ring, which ring optionally bears alkyl or aryl substituents; and/or

R₁″ and R₂″ are alkyl groups connected such that they together comprises a 3, 4, 5, 6 or 7-membered ring, which ring optionally bears alkyl or aryl substituents; and/or

R₁′ and Y are connected such that they together comprises a 5, 6 or 7-membered ring; and/or

R₁′ and Y are connected such that they together comprises a 5, 6 or 7-membered ring; and/or

R₂′ and Z are connected such that they together comprises a 5, 6 or 7-membered ring; and/or

R₁″ and W are connected such that they together comprises a 5, 6 or 7-membered ring; and/or

R₂″ and X are connected such that they together comprises a 5, 6 or 7-membered ring; and

A is an anion.

Active compounds for use in the present invention include but are not limited to compounds described in U.S. Pat. Nos. 7,906,500 and 8,158,674 to Detty et al., in A. Orchard et al., Bioorganic & Med. Chem. 20, 4290-4302 (2012), the disclosures of which are incorporated by reference herein in their entirety.

In some embodiments, active compounds of the present invention are compounds of Formula Ia:

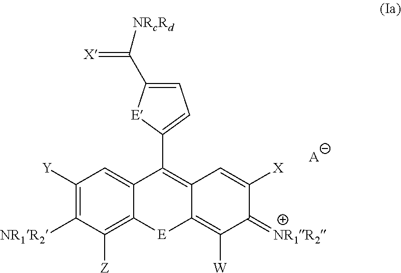

wherein:
E is S or Se;
E′ is O, S, NH, or NRₑ wherein Rₑ is C1 to C6, linear or branched, alkyl (preferably, E′ is S);
X′ is O or S (preferably O);
W, X, Y, and Z, and R₁′, R₂′, R₁″ and R₂″, are as described in connection with Formula I above; and
R_{c} and R_{d} are each independently H or C1 to C6, linear or branched, alkyl, or R_{c} and R_{d} together form with N a 3, 4, 5 6, or 7 membered ring (e.g., R_{c} and R_{d} together form C2 to C6 alkylene); and
A is an anion.

In some embodiments, active compounds of the present invention are compounds of Formula Ib:

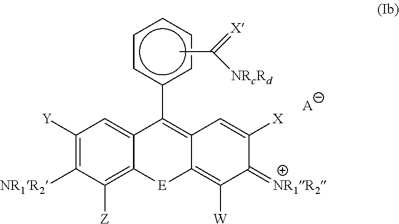

wherein:
E is S or Se;
X′ is O or S (preferably O);
W, X, Y, and Z, and R₁′, R₂′, R₁″ and R₂″, and R_{c} and R_{d}, are as described in connection with Formula I above;
the group —C(═X′)(—NR_{c}R_{d}) can be in the 2—(ortho), 3—(meta), or 4—(para) position; and
A is an anion.

In some embodiments, active compounds of the present invention are compounds of Formula IIa:

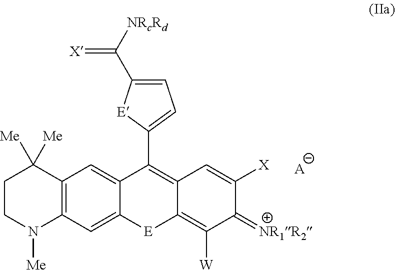

wherein:
E is S or Se;

E′ is O, S, NH, or NRₑ, wherein Rₑ is C1 to C6, linear or branched, alkyl (preferably, E′ is S);
X′ is O or S (preferably O);
W and X, R₁″ and R₂″, and R_{c} and R_{d}, are as described in connection with Formula I above; and
A is an anion.

In some embodiments, active compounds of the present invention are compounds of Formula IIb:

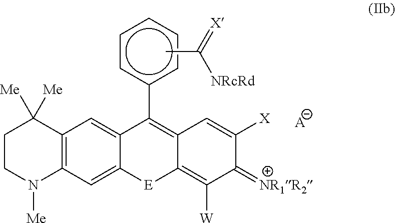

wherein:
E is S or Se;
X′ is O or S (preferably O);
W and X, R₁″ and R₂″, and R_{c} and R_{d}, are as described in connection with Formula I above;
the group —C(═X′)(—NR_{c}R_{d}) can be in the 2—(ortho), 3—(meta), or 4—(para) position; and
A is an anion.

In some embodiments, active compounds of the present invention are compounds of Formula IIIa:

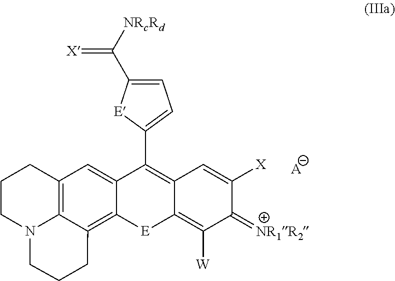

wherein:
E is S or Se;
E′ is O, S, NH, or NRₑ, wherein Rₑ is C1 to C6, linear or branched, alkyl (preferably, E′ is S);
X′ is O or S (preferably O);
W and X, R₁″ and R₂″, and R_{c} and R_{d}, are as described in connection with Formula I above; and
A is an anion.

In some embodiments, active compounds of the present invention are compounds of Formula IIIb:

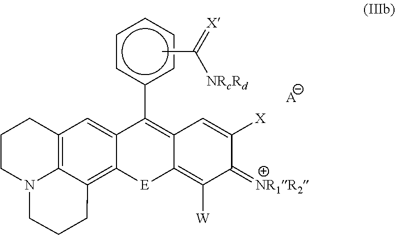

wherein:
E is S or Se;
X′ is O or S (preferably O);
W and X, R₁″ and R₂″, and R_{c} and R_{d}, are as described in connection with Formula I above;
the group —C(═X′)(—NR_{c}R_{d}) can be in the 2—(ortho), 3—(meta), or 4—(para) position; and
A is an anion.

In some embodiments, active compounds of the present invention are compounds of Formula IVa:

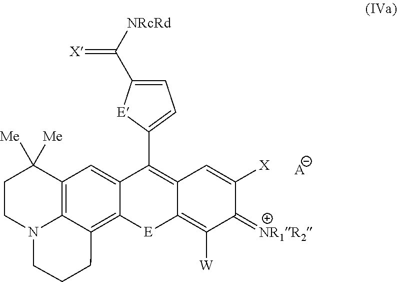

wherein:
E is S or Se;
E′ is O, S, NH, or NRₑ, wherein Rₑ is C1 to C6, linear or branched, alkyl;
X′ is O or S (preferably O);
W and X, R₁″ and R₂″, and R_{c} and R_{d}, are as described in connection with Formula I above; and
A is an anion.

In some embodiments, active compounds of the present invention are compounds of Formula IVb:

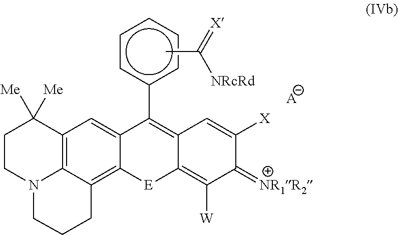

wherein:
E is S or Se;
X′ is O or S (preferably O);
W and X, R₁″ and R₂″, and R_{c} and R_{d}, are as described in connection with Formula I above;
the group —C(═X′)(—NR_{c}R_{d}) can be in the 2—(ortho), 3—(meta), or 4—(para) position; and
A is an anion.

In some embodiments, active compounds of the present invention are compounds of Formula Va:

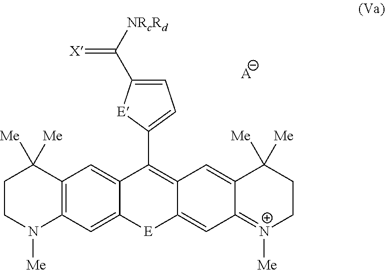

wherein:
E is S or Se;
E′ is O, S, NH, or NRₑ, wherein Rₑ is C1 to C6, linear or branched, alkyl (preferably E′ is S);
X′ is O or S (preferably O);
R_{c} and R_{d} are as described in connection with Formula I above; and
A is an anion.

In some embodiments, active compounds of the present invention are compounds of Formula Vb:

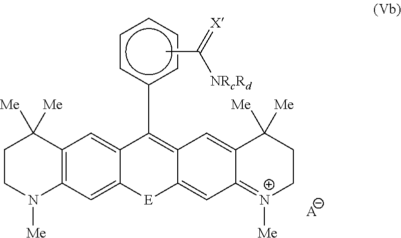

wherein:
E is S or Se;
X′ is O or S (preferably O);
R_{c} and R_{d} are as described in connection with Formula I above;
the group —C(═X′)(—NR_{c}R_{d}) can be in the 2—(ortho), 3—(meta), or 4—(para) position; and
A is an anion.

In some embodiments, active compounds of the present invention are compounds of Formula VIa:

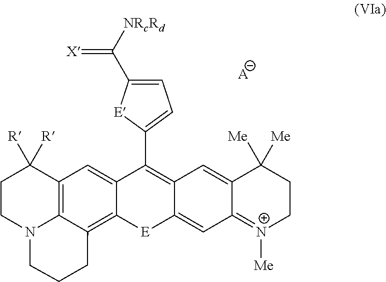

wherein:
E is S or Se;
E′ is O, S, NH, or NRₑ, wherein Rₑ is C1 to C6, linear or branched, alkyl (preferably E′ is S);
X′ is O or S (preferably O);
R_{c} and R_{d} are as described in connection with Formula I above; and
each R′ is independently H or C1 to C6, linear or branched, alkyl (e.g., methyl, ethyl); and
A is an anion.

In some embodiments, active compounds of the present invention are compounds of Formula VIb:

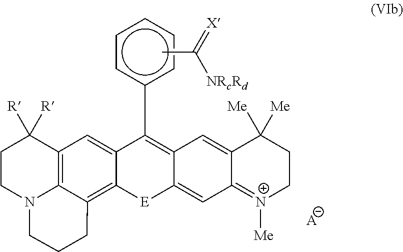

wherein:
E is S or Se;
X′ is O or S (preferably O);
R_{c} and R_{d} are as described in connection with Formula I above;
each R′ is independently H or alkyl (e.g., methyl, ethyl);
the group —C(═X′)(—NR_{c}R_{d}) can be in the 2—(ortho), 3—(meta), or 4—(para) position; and
A is an anion.

In some embodiments, active compounds of the present invention are compounds of Formula VIIa:

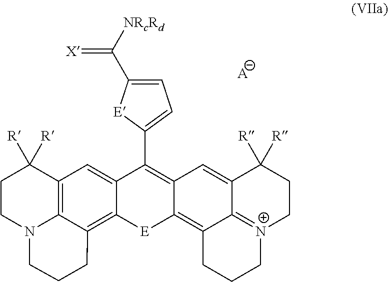

wherein:
E is S or Se;
E is O, S, NH, or NRₑ, wherein Rₑ is C1 to C6, linear or branched, alkyl (preferably, E′ is S);
X′ is O or S (preferably O); and
each R′ is independently H or alkyl (e.g., methyl, ethyl);
each R″ is independently H or alkyl (e.g., methyl, ethyl);
R_{c} and R_{d} are as described in connection with Formula I above; and
A is an anion.

In some embodiments, active compounds of the present invention are compounds of Formula VIIb:

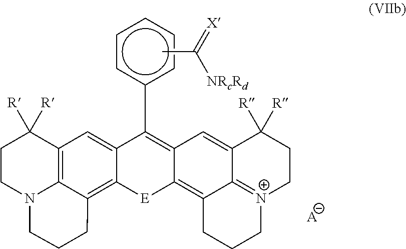

wherein:
E is S or Se;
X′ is O or S (preferably O);
each R′ is independently H or alkyl (e.g., methyl, ethyl);
each R″ is independently H or alkyl (e.g., methyl, ethyl);
R_{c} and R_{d} are as described in connection with Formula I above; and
the group —C(═X′)(—NR_{c}R_{d}) can be in the 2—(ortho), 3—(meta), or 4—(para) position; and
A is an anion.

Active compounds for use in the present invention are made in accordance with the techniques described herein, and/or known techniques such as described in U.S. Pat. Nos. 7,906,500 and 8,158,674 to Detty et al. and in A. Orchard et al., Bioorganic & Med. Chem. 20, 4290-4302 (2012), and/or variations thereof which will be apparent to those skilled in the art based upon the present disclosure.

### 3. Methods.

As noted above, the present invention provides a method of selectively depleting pathogenic T lymphocytes from a blood cell composition, comprising: (a) combining said cell composition (generally a biological fluid) ex vivo with an active compound as described herein in an effective amount, and then (b) irradiating said cells with light (preferably ultraviolet light, and particularly UV-A) ex vivo for a time and at an intensity sufficient to selectively kill pathogenic T lymphocytes in said cell composition.

Photopheresis apparatus and methods useful for carrying out the present invention include, but are not limited to, those described in U.S. Pat. Nos. 7,476,209, 5,951,509; 5,985,914; 5,984,887, 4,464,166; 4,428,744; 4,398,906; 4,321,919; and in U.S. Patent Application Publication Nos. US 2014/0081193 and 2012/0197419, the disclosures of all of which are expressly incorporated herein by reference. Examples of commercial photopheresis apparatus that may be used to carry out the present invention include, but are not limited to,

Biological fluids on which the methods of the invention may be carried out will depend upon the condition being treated and the system or apparatus in which the method is carried out. In general, the biological fluid can be: (i) whole blood, (ii) a white blood cell-containing fraction of whole blood (e.g. a fraction produced by centrifugation of whole blood to separate red blood cells, the fraction optionally also containing other leukocytes such as neutrophils, platelets, blood plasma, etc., including but not limited to a buffy coat blood fraction), or (iii) a hematopoietic stem cell-containing fraction of blood or tissue (e.g., bone marrow stem cells, peripheral blood stem cells, amniotic fluid stem cells, or umbilical cord blood cells).

Pathogenic T lymphocytes in the cell composition/biological fluid are, in some embodiments, alloreactive T-lymphocytes (e.g. in a blood cell composition collected from a hematopoietic stem cell transplant donor, or solid organ transplant recipient).

Pathogenic T lymphocytes in the cell composition/biological fluid are, in other embodiments, autoreactive T-lymphocytes (e.g., in a blood cell composition collected from a patient afflicted with an autoimmune disease).

Pathogenic T lymphocytes in the cell composition/biological fluid are, in still other embodiments, malignant T-lyphocytes (e.g., in a blood cell composition collected from a patient afflicted with T-cell lymphoma).

In some embodiments, (e.g., where the pathogenic T-lymphocytes are autoreactive T-lymphocytes), the blood cell composition/biological fluid can be collected from a subject afflicted with an autoimmune disease, examples of which include but are not limited to graft versus host disease (GVHD), scleroderma, atopic dermatitis, epidermolysis bullosa acquisita, lichen planus, lupus erythematosus, pemphigus vulgaris, Crohn disease, type 1 diabetes, psoriasis, rheumatoid arthritis, multiple sclerosis, nephrogenic systemic fibrosis/nephrogenic fibrosing dermopathy, and scleromyxedema.

The amount of active agent administered to the biological fluid will vary depending upon factors such as the particular type of biological fluid used and the particular condition being treated. In general, the active agent is combined with the biological fluid in an amount of 1, 10, or 50 milligrams per liter, up to 400, 600, 800, or 1000 milligrams per liter.

In general, the irradiating step is carried out with an artificial source of ultraviolet light (e.g., UV-A) included the particular apparatus employed. The irradiating step may be carried out in a “batch” fashion, or carried out continuously under sterile conditions in an enclosed fluid circuit containing said blood cell composition, again provided by the particular apparatus employed.

The time and intensity, or effective amount of, light energy that is delivered to the biological fluids may be determined using the methods and systems described in U.S. Pat. No. 6,219,584, the disclosure of which is incorporated herein by reference in its entirety.

Once irradiation is completed, the cells are administered (e.g., by intraveneous injection) to a subject in need thereof in accordance with known techniques. In some embodiments, the subject is a subject in need of a hematopoietic stem cell transplant (in which case the recipient is a different subject than the donor); in some embodiments the subject is afflicted with a T-cell lymphoma; in some embodiments, the subject is a subject afflicted with an autoimmune disease.

The present invention is explained in greater detail in the following non-limiting Examples.

### EXAMPLES

It has previously been demonstrated that when using a photosensitive agent, prolonged intracellular resident times were associated with non-selective depletion of susceptible lymphocyte subsets.⁶ Dibromorhodamine-123 is a photosensitive agent that is highly dependent on P-glycoprotein (P-gp) for cell extrusion, and cells that express low P-gp activity are susceptible to increased intracellular photosensitizer accumulation. Consequently, lymphocyte subsets with low P-gp activity, such as B cells, and CD4+ and memory T cells, are disproportionately depleted when using this agent. In the clinical setting of immune therapy, the use of dibromorhodamine-123 has resulted in the non-selective depletion of lymphocytes important for normal immune responses, and poor patient outcomes.⁷

P-glycoprotein (also known as MDR1 or ABCB1) is a member of the ATP-binding cassette (ABC) superfamily and was the first efflux protein identified and associated with multidrug resistance in cancer chemotherapy.⁸ P-gp is able to transport a diverse array of anticancer drugs including anthracyclines, vinca alkaloids, taxanes, epipodophyllotoxins, and agents such as mitomycin C, dactinomycim, and trimetrexate.⁹⁻¹¹ Since the discovery of verapamil as an inhibitor of P-gp, many approaches to the development of inhibitors/modulators of P-gp have been examined.^{12,13}

It has recently been demonstrated that simple substitutions in a series of chalcogenorosamine/rhodamine structures can create molecules that possess a high affinity for P-gp and are either highly stimulating or inhibiting for ATPase activity.¹⁴ This work has demonstrated that specific tertiary amide and thioamide group substitutions dictate ATPase stimulation. This amide/thioamide modification effectively controls the rate of transport of rhodamine derivatives in both absorptive and secretory directions in the cell.¹⁵

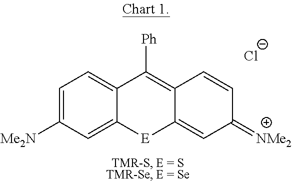

Selenorosamine and selenorhodamine analogues of the chalcogenorosamine/rhodamines are more effective photosensitizers in vitro than lighter chalcogen analogues for photodynamic therapy of both chemoresponsive¹⁶ and P-gp-expressing, drug-resistant¹⁷ cancer cells. This, perhaps, is a consequence of the increased quantum yields for the generation of singlet oxygen [Φ(¹O₂)] in the selenium-containing analogues relative to the sulfur-containing analogues.^{16,18} Studies of whole-cell cytochrome c oxidase activity suggest that the mitochondria are targets for the chalcogenorosamine photosensitizers TMR-S and TMR-Se (Chart 1). Irradiation of TMR-S- or TMR-Se-treated cells gives light fluence-dependent inhibition of cytochrome c oxidase activity.¹⁶

The ability of the chalcogenorosamine/rhodamines to modulate P-gp activity and the ability to target the mitochondria provide the basis for a new approach to ECP. To provide an example, we evaluated the 24 photosensitive chalcogenorhodamines shown in Chart 2 for their potential application in targeting reactive and malignant T cells. The varied thioamide scaffolds of Chart 2 have inhibited ATPase activity in P-gp while the amide scaffolds have stimulated ATPase activity. As an alternative to binding DNA, the combination of mitochondrial-specific agents and control of P-gp stimulation gave candidates with improved selectivity and reduced toxicity of the photosensitizers.

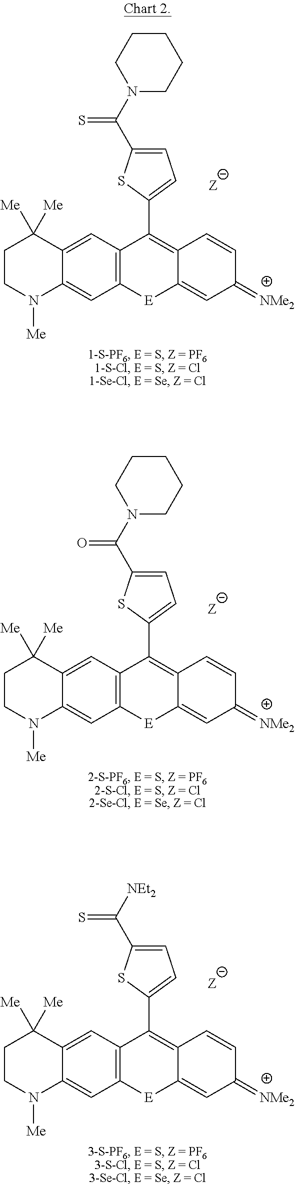

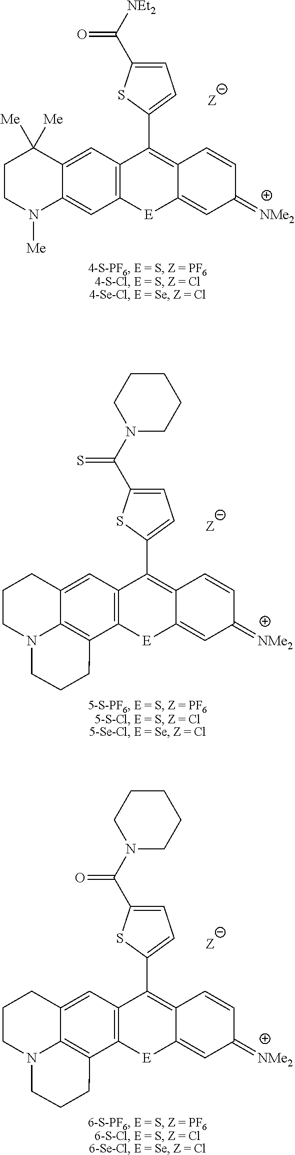

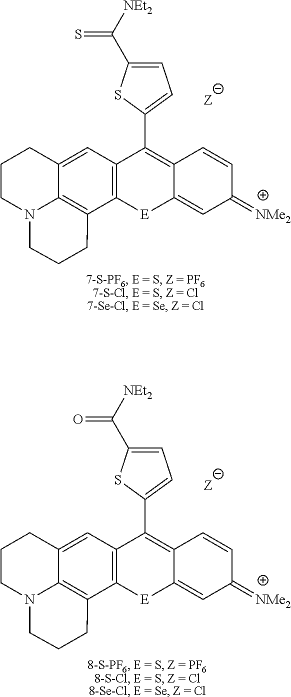

Results. Compounds 1-4 incorporate a trimethyltetrahydroquinoline group for one of the rhodamine amino substituents (Chart 3). Compounds 5-8 incorporate an azadecalin substituent for one of the rhodamine amino substituents. The fused aniline equivalent of the azadecalin substitution is known as julolidine (Chart 3) and compounds 5-8 are referred to as “julolidyl” rhodamines herein. Similarly, compounds incorporating the trimethyltetrahydroquinoline group are referred to as “half-julolidyl” rhodamines herein.

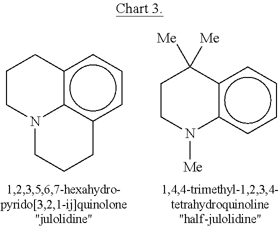

Synthesis of Thiorhodamine analogues 1-S-8-S. The thiorhodamines 1-S-8-S as the PF₆⁻ salts (Chart 1) were prepared by literature procedures.¹⁵ The PF₆⁻ salts were converted to the chloride salts using Amberlite IRA-400 chloride ion-exchange resin.

Synthesis of Selenorhodamine analogues 1-Se—Cl-8-Se—Cl. The key intermediate for the synthesis of photosensitizers 1-Se—Cl-4-Se—Cl is selenoxanthone 9 (Scheme 1). The synthesis of 9 begins with amide 10.¹⁵ Directed ortho-lithiation of 20 in THF at −78° C. with sec-butyllithium and N,N,N′,N′-tetramethylethylenediamine (TMEDA) was followed immediately by the addition of 3-dimethylaminophenyl diselenide (11)^{19,20} at −78° C. The immediate addition was necessary to minimize the amount of self-condensed side product formation that has been seen in similar reactions. The isolated yield of unsymmetrical diaryl selenide 12 was 46%. Subsequent cyclization of 12 with POCl₃ in acetonitrile¹⁹ gave the desired selenoxanthone 9 in 96% isolated yield.

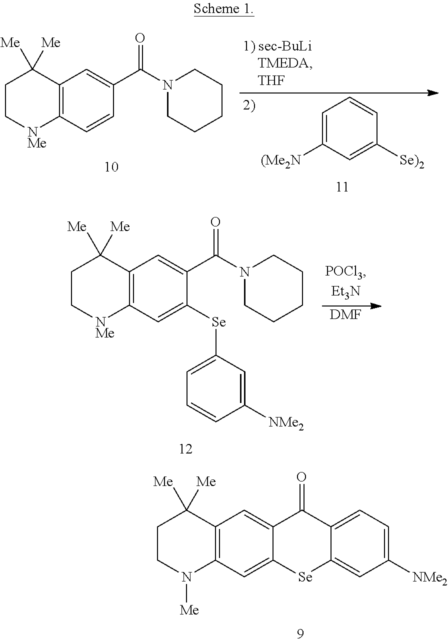

Thioamide 13 was prepared in 94% isolated yield from thiophene-2-carboxaldehyde under Wilgerodt-Kindler conditions with elemental sulfur and piperidine.^{14,15} Deprotonation of 13 with lithium diisopropylamide (LDA) occurred from the sterically least hindered 5-position to give N-piperidyl 5-lithio-2-thiocarboxythiophene (14), which was added to selenoxanthone 9 to give selenorhodamine thioamide 15 in 81% isolated yield following workup with aqueous HPF₆ (Scheme 2). Trifluoroacetic anhydride was added to CH₂Cl₂ solutions of thioamide 15 to give the corresponding amide 16 in 11% isolated yield.¹⁹ Compounds 15 and 16 were converted to the corresponding chloride salts, 1-Se—Cl and 2-Se—Cl, respectively, with Amberlite IRA-400 chloride ion-exchange resin.

Willgerodt-Kindler oxidation of thiophene-2-carboxaldehyde with elemental sulfur and diethylamine gave thioamide 17 in 49% isolated yield.²¹ Deprotonation of 17 with LDA gave the 2-thienyl anion 18, which was then added to a solution of selenoxanthone 9 (Scheme 2). Workup with aqueous HPF₆ gave the diethyl thioamide-containing photosensitizer 19 in 34% isolated yield. Trifluoroacetic anhydride was added to CH₂Cl₂ solutions of thioamide 19 to give the corresponding amide 20 in 44% isolated yield.¹⁹ Compounds 19 and 20 were converted to the corresponding chloride salts, 3-Se—Cl and 4-Se—Cl, respectively, with Amberlite IRA-400 chloride ion-exchange resin.

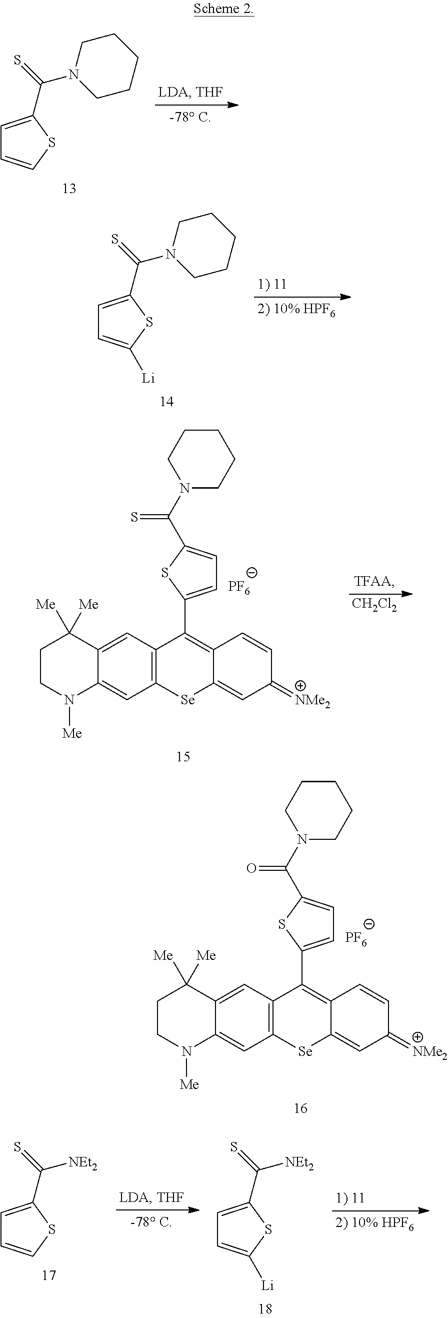

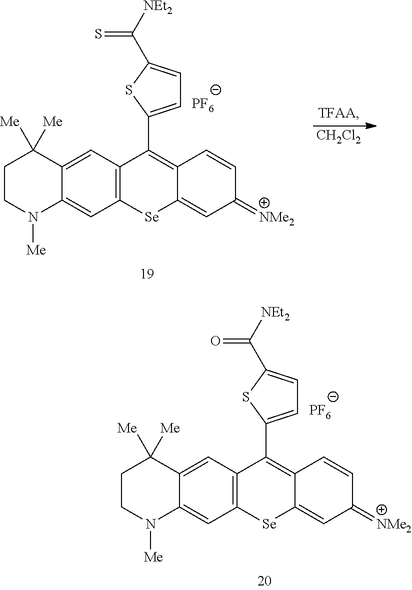

The starting point for the synthesis of the julolidyl selenoxanthylium photosensitizers 5-Se—Cl-8-Se—Cl was the known selenoxanthone 21 (Scheme 3).²² The synthesis of thioamide-containing selenorhodamine 22 by the addition of anion 14 to selenoxanthone 21 followed by workup with 10% HPF₆ has been reported and gives 22 in 34% isolated yield.¹⁴ Trifluoroacetic anhydride was added to CH₂Cl₂ solutions of thioamide 22 to give the corresponding amide 23 in 54% isolated yield.¹⁹ Similarly, the addition of anion 18 to selenoxanthone 21 followed by workup with 10% HPF₆ gave thioamide-containing selenorhodamine 24 in 84% isolated yield. Trifluoroacetic anhydride was added to CH₂Cl₂ solutions of thioamide 24 to give the corresponding amide 25 in 55% isolated yield.¹⁹ Compounds 22-25 were converted to the corresponding chloride salts, 5-Se—Cl-8-Se—Cl, respectively, with Amberlite IRA-400 chloride ion-exchange resin.

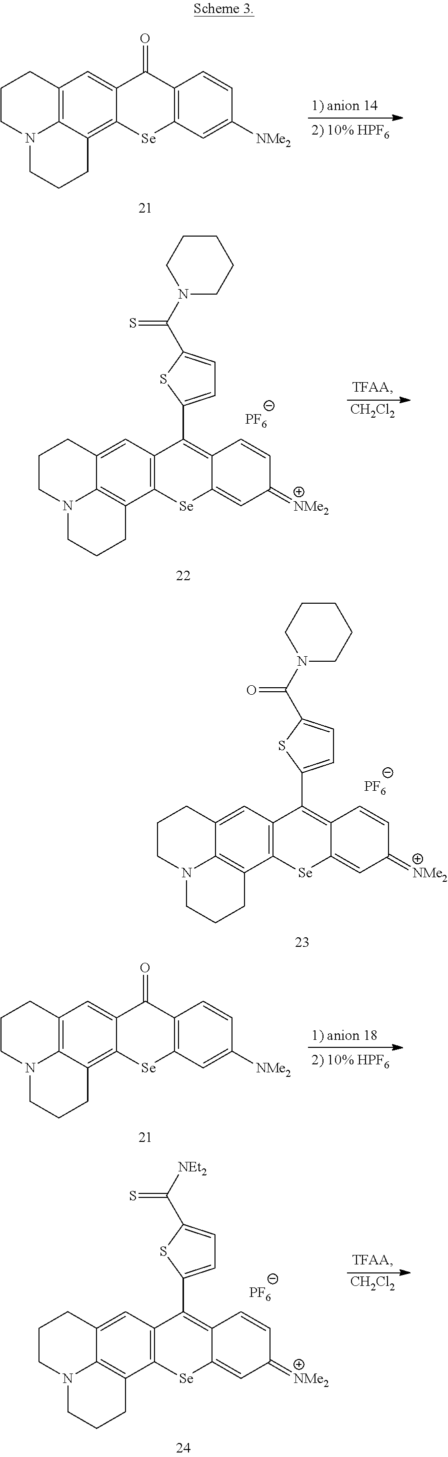

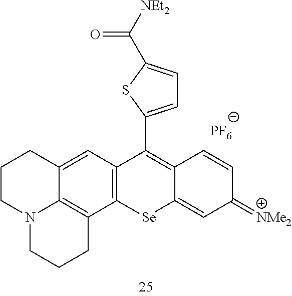

Spectral data and Quantum Yields for the Generation of Singlet Oxygen and Fluorescence. The electronic spectra of the thiorhodamines based on scaffolds 1-8 were similar with absorption maxima (λₘₐₓ) between 606 and 614 nm in MeOH as were the electronic spectra of the selenorhodamines based on scaffolds 1-8 with absorption maxima (λₘₐₓ) between 617 and 622 nm in MeOH. Molar extinction coefficients (c) for the chalcogenorhodamines were between 7.4×10⁴ and 1.3×10⁵ M⁻¹ cm⁻¹ (Table 1). In general, values of λₘₐₓ for the julolidyl photosensitizers were 3-5 nm longer than values of λₘₐₓ for the corresponding half-julolidyl photosensitizers.

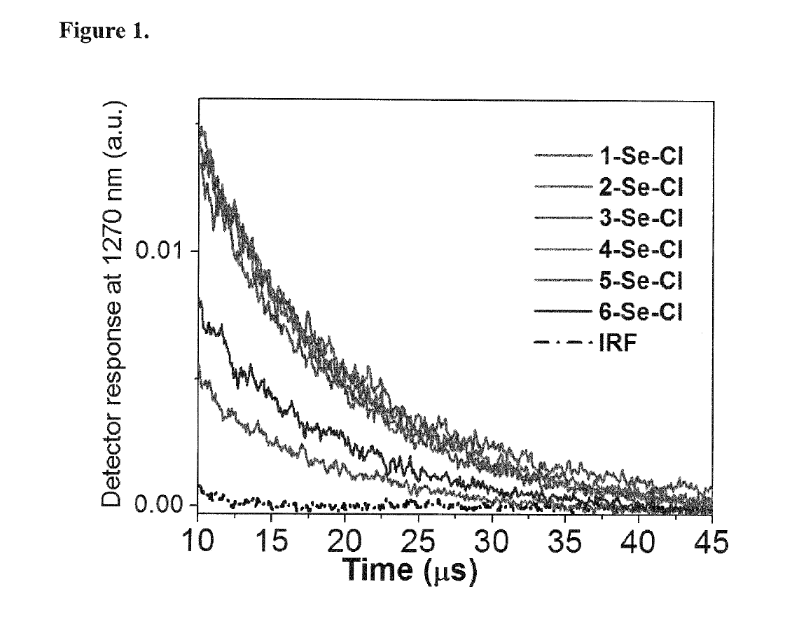

Quantum yields for the generation of ¹O₂ [Φ(¹O₂)] by 1-Se—Cl-6-Se—Cl were evaluated using time-resolved spectroscopy of ¹O₂ phosphorescence (FIG. 1).²³ We employed TMR-Se (Chart 1) as a reference [Φ(¹O₂)=0.87].¹⁸ Values of Φ(¹O₂) for 1-Se—Cl-6-Se—Cl were obtained by comparing decays of ¹O₂ phosphorescence sensitized by 1-Se—Cl-6-Se—Cl in air-saturated MeOH solutions (Table 1).

TABLE 1
Spectral and photophysical properties for chalcogenorhodamine photosensitizers 1-8.

| | λₘₐₓ, | ε, | λ_{EM,} | Φ_{F}, | R.F., | Φ(¹O₂), | |
|---|---|---|---|---|---|---|---|
| Compd | nm^{a} | M⁻¹ cm^{-1a} | nm^{b} | MeOH | a.u.^{b,c} | MeOH | log P |
| 1-S—PF₆ | 610^{d} | 9.8 × 10⁴ | 630 | 0.07 ± 0.01 | — | — | 1.2 |
| 1-S—Cl | 608 | 9.8 × 10⁴ | 630 | 0.07 ± 0.01 | 0.40 | — | 1.92 ± 0.08 |
| 1-Se—Cl | 617 | 1.2 × 10⁵ | 640 | 0.008 ± 0.001 | 0.037 | 0.44 ± 0.03 | 1.61 ± 0.06 |
| 2-S—PF₆ | 609^{d} | 1.2 × 10⁵ | 632 | 0.09 ± 0.01 | — | — | 1.2 |
| 2-S—Cl | 606 | 1.2 × 10⁵ | 632 | 0.09 ± 0.01 | 0.80 | — | 2.26 ± 0.04 |
| 2-Se—Cl | 618 | 7.4 × 10⁴ | 638 | 0.009 ± 0.001 | 0.038 | 0.48 ± 0.03 | 2.23 ± 0.04 |
| 3-S—PF₆ | 609^{d} | 1.1 × 10⁵ | 630 | 0.07 ± 0.01 | — | — | 1.7 |
| 3-S—Cl | 608 | 1.1 × 10⁵ | 630 | 0.07 ± 0.01 | 0.57 | — | 1.98 ± 0.06 |
| 3-Se—Cl | 617 | 8.6 × 10⁴ | 638 | 0.008 ± 0.002 | 0.037 | 0.54 ± 0.03 | 2.41 ± 0.04 |
| 4-S—PF₆ | 608^{d} | 1.2 × 10⁵ | 630 | 0.09 ± 0.01 | — | — | 1.5 |
| 4-S—Cl | 606 | 1.2 × 10⁵ | 630 | 0.09 ± 0.01 | 0.56 | — | 2.16 ± 0.02 |
| 4-Se—Cl | 617 | 1.3 × 10⁵ | 638 | 0.009 ± 0.001 | 0.037 | 0.50 ± 0.03 | 2.26 ± 0.04 |
| 5-S—PF₆ | 608 | 9.4 × 10⁴ | 634 (632) | 0.28 ± 0.01 | — | — | 1.7 |
| 5-S—Cl | 614 | 1.0 × 10⁵ | 634 (632) | 0.30 ± 0.01 | 0.84 | — | 2.97 ± 0.06 |
| 5-Se—Cl | 622 | 1.3 × 10⁵ | 648 (647) | 0.009 ± 0.002 | 0.040 | 0.13 ± 0.02 | 3.00 ± 0.02 |
| 6-S—PF₆ | 612^{d} | 1.0 × 10⁵ | 632 (630) | 0.36 ± 0.01 | — | — | 1.9 |
| 6-S—Cl | 613 | 1.0 × 10⁵ | 632 (630) | 0.36 ± 0.01 | 1.00 | — | 2.69 ± 0.02 |
| 6-Se—Cl | 622 | 1.1 × 10⁵ | 646 (645) | 0.011 ± 0.002 | 0.072 | 0.23 ± 0.03 | 2.74 ± 0.02 |
| 7-S—PF₆ | 611^{d} | 1.1 × 10⁵ | 634 | — | — | — | 2.7 |
| 7-S—Cl | 614 | 1.1 × 10⁵ | 634 | — | 0.63 | — | 2.55 ± 0.02 |
| 7-Se—Cl | 622 | 8.6 × 10⁴ | 644 | — | 0.053 | — | 3.00 ± 0.02 |
| 8-S—PF₆ | 611^{d} | 1.0 × 10⁵ | 634 | — | — | — | 1.4 |
| 8-S—Cl | 614 | 1.0 × 10⁵ | 634 | — | 0.85 | — | 2.65 ± 0.02 |
| 8-Se—Cl | 622 | 1.3 × 10⁵ | 644 | — | 0.069 | — | 2.87 ± 0.01 |

^{a}In MeOH.
^{b}In 1% BSA, 10% MeOH in pH 7.4 phosphate buffer with excitation at 532 nm. Values in parentheses are in MeOH.
^{c}Relative fluorescence (R.F.) in arbitrary units (a.u.) at λ_{EM} with excitation at 532 nm.
^{d}In CH₂Cl₂.

Values of Φ(¹O₂) ranged from 0.13±0.03 for 5-Se—Cl to 0.54±0.03 for 3-Se—Cl. Values of Φ(¹O₂) for the julolidy thioamide 5-Se—Cl and amide 6-Se—Cl were significantly lower (p<0.0001, Student t-test for pair-wise comparisons) relative to the half-julolidyl thioamides 1-Se—Cl and 3-Se—Cl and amides 2-Se—Cl and 4-Se—Cl.

Quantum yields for fluorescence (Φ_{F}) for chalcogenorhodamines 5-S—PF₆, 5-S—Cl, 5-Se—Cl, 5-S—PF₆, 5-S—Cl, and 5-Se—Cl were determined in MeOH using TMR-S (Chart 1) as a reference [ _{F}=0.44].^{17,18} As shown in Table 1, values of Φ_{F} were identical within experimental error for the chloride and PF₆⁻ salts of both the 5-S and 6-S series. Values of Φ_{F} were roughly 30-fold higher for the sulfur analogues 5-S—Cl and 6-S—Cl relative to the selenium analogues 5-Se—Cl and 6-Se—Cl, respectively. Values of λ_{EM} and relative fluorescence values (R.F.) in 1% bovine serum albumin (BSA) and 10% MeOH in pH 7.4 phosphate buffer are compiled in Table 1 for samples with an optical density of 0.10 at 532 nm, which was the excitation wavelength.

Measurement of n-octanol/water partition coefficients. Experimental values of the n-octanol/water partition coefficient (log P) for chalcogenorhodamine photosensitizers 1-8 were measured using the “shake flask” method. A saturated n-octanol solution of selenorhodamine was shaken with an equal volume of phosphate buffered saline (PBS) at pH 7.4 and the concentrations in the two layers were determined spectrophotometrically. Values of log P are compiled in Table 1.

Cellular Uptake and Extrusion of Chalcogenorhodamine Photosensitizers. Earlier studies have shown that for chalcogenorhodamines with amide or thioamide substituents, the amide-substituted derivatives were ATPase activating in P-gp while the thioamide-substituted derivatives were ATPase deactivating.^{14,15} Consistent with these observations, we found that the thioamide analogues of the chalcogenorhodamines (scaffolds 1, 3, 5, and 7) were associated with prolonged intracellular retention compared to their amid pairs (scaffolds 2, 4, 6, and 8). Cellular uptake of the chalcogenorhodamine photosensitizers was measured after a 20 minute exposure to 2.5×10⁻⁷ M photosensitizer in malignant T cells (HUT-78 cells, Table 2). Transmembrane movement of the photosensitizers in the secretory direction (basolateral to apical) was measured after an 18-h extrusion period. As described above, the Se-containing molecules fluoresced at a lower intensity relative to the S-containing molecules. Higher mean fluorescence intensities (MFIs) were noted with the thioamide analogues compared to their amide pairs (p<0.05, Student t-test for pair-wise comparisons) in scaffolds 1-8 after both the uptake and extrusion periods. Overall, the PF₆⁻ salts were more slowly extruded from cells relative to the chloride salts of the amide, with a greater difference in the extrusion kinetics noted between the thiorhodamine PF₆ salts compared to the selenorhodamine chloride salts.

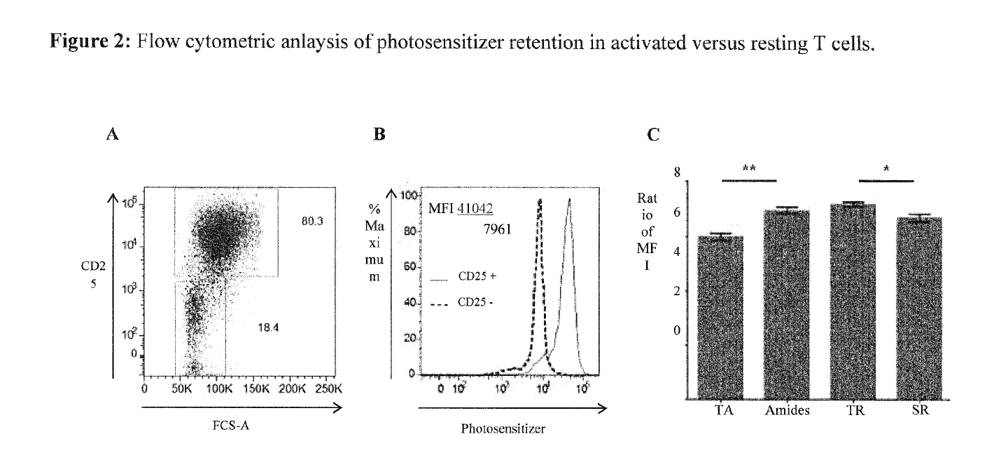

Activated and resting T cells can be accurately differentiated by CD25 expression as shown in FIG. 2A.²⁴ All chalcogenorhodamine analogues evaluated demonstrated higher uptake within activated and malignant T cells. To evaluate for selective uptake in activated T cells, SEB stimulated human peripheral blood mononuclear cells (PBMC) were washed and suspended at a concentration of 2×10⁶ cells/mL in 2.5×10⁻⁷ M of photosensitizer for 20 minutes, followed by suspension in photosensitizer-free media for 30 minutes. Fluorescence intensity of the photosensitizers was 5 to 7-fold higher in CD25+ T cells compared to CD25− T cells (FIGS. 2B and 2C; mean MFI 6.46; range 5.52 to 7.46), and was in proportion to the extent of P-gp stimulation. The amide analogues were associated with a significantly greater selective accumulate in activated T cells compared to the thioamide analogues, and the thiorhodamine scaffolds were associated with the highest retention differential (FIG. 2C). Preferential uptake was increased at higher concentrations (data not shown). These results demonstrate that P-gp stimulation promotes selective accumulation of photosensitizer in activated T cells, and therefore may improve the selectivity of phototherapy.

TABLE 2
Uptake and extrusion kinetics of chalcogenorhodamine
thioamide and amide analogues 1-8.^{a}

| Thioamide Analogues | | | Amide Analogues | | | |
|---|---|---|---|---|---|---|
| Compd | Uptake MFI | % Extrusion ^{c} | Compd | Uptake MFI | | % Extrusion ^{c} |
| 1-S—PF6 | 30229 (±1124) | 84.16 (±3.00) | 2-S—PF6 | 71978 | (±10596) | 91.79 (±1.16) |
| 1-S—CL | 30070 (±2481) | 84.97 (±2.87) | 2-S—CL | 50993 | (±3815) | 92.70 (±0.74) |
| 1-Se—CL | 6162 (±633) | 90.06 (±1.95) | 2-Se—CL | 6536 | (±641) | 92.17 (±1.09) |
| 3-S—PF6 | 45685 (±2726) | 84.91 (±1.57) | 4-S—PF6 | 66500 | (±7683) | 92.20 (±0.55) |
| 3-S—CL | 38237 (±2684) | 86.81 (±0.89) | 4-S—CL | 64332 | (±6630) | 92.08 (±0.49) |
| 3-Se—CL | 5480 (±666) | 88.65 (±1.21) | 4-Se—CL | 5209 | (±112) | 92.24 (±0.69) |
| 5-S—PF6 | 80192 (±4122) | 76.52 (±4.79) | 6-S—PF6 | 114190 | (±9447) | 82.54 (±1.17) |
| 5-S—CL | 76661 (±5618) | 76.13 (±5.21) | 6-S—CL | 122500 | (±6436) | 82.10 (±1.17) |
| 5-Se—CL | 12814 (±1256) | 85.03 (±2.90) | 6-Se—CL | 14910 | (±2041) | 88.39 (±1.13) |
| 7-S—PF6 | 87456 (±10089) | 73.25 (±2.15) | 8-S—PF6 | 128000 | (±8916) | 79.40 (±1.56) |
| 7-S—CL | 63950 (±5511) | 74.88 (±3.62) | 8-S—CL | 114750 | (±7653) | 78.55 (±2.35) |
| 7-Se—CL | 14935 (±1810) | 81.23 (±2.56) | 8-Se—CL | 19365 | (±856) | 83.05 (±1.78) |

^{a}Uptake and extrusion experiments were performed with 2.5 × 10⁻⁷ M photosensitizer. Uptake mean fluorescent intensity (MFI) was measured after 20 minutes of photosensitizer exposure.
^{b} Ratio of MFI of CD25+ and CD25− T cells.
^{c} Percent extrusion was measured in HUT-78 cells and was calculated as the percent change in MFI after an 18 hour extrusion period.
^{d} Percent of HUT-78 cells that were photosensitizer free after an 18 hour photosensitizer extrusion period. Details for methods are provided in Experimental Section. Error limits represent ±SE.

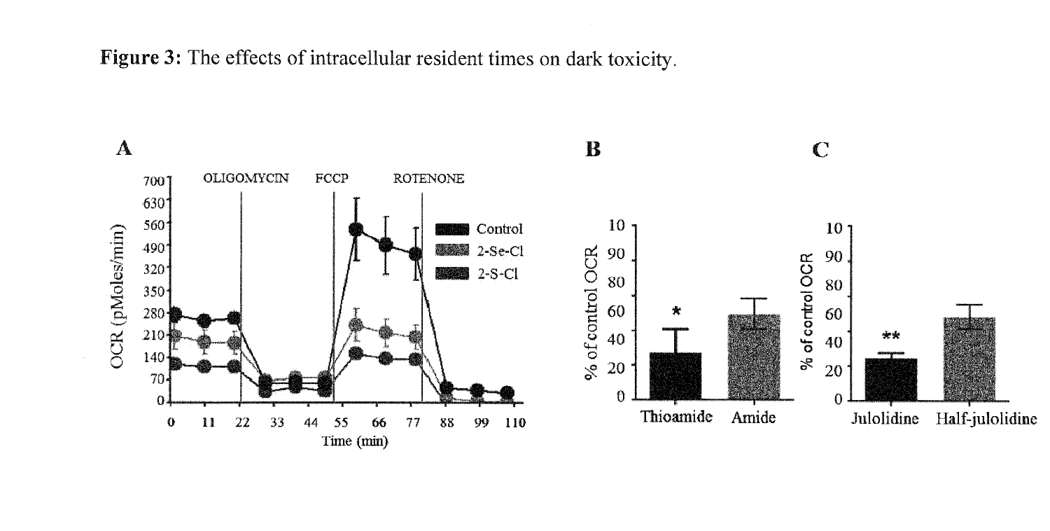

Dark Toxicity of Chalcogenorhodamine Photosensitizers. To determine the degree of dark toxicity of the chalcogenorhodamine photosensitizers, we measured the bioenergetics profiles of resting T cells after a 20 minute uptake of 5.0×10⁻⁷ M photosensitizer followed by a 4-h extrusion period in a basal state, and after the addition of oligomycin (to block ATP synthesis), FCCP (to uncouple ATP synthesis from the electron transport chain), and rotenone (to block complex I of the electron transport chain). For each chalcogenorhodamine series, results were compared to the bioenergetics profiles of non-exposed resting T cells. For this analysis, we defined the percent expected O₂ consumption rate (OCR)=OCR of photosensitizer-exposed cells/OCR of control (photosensitizer-free) cells (FIG. 3A for cells exposed to 2-S—Cl or 2-Se—Cl). In general, we found that the dark toxicity profiles were closely associated with the extrusion kinetics of the chalcogenorhodamine photosensitizers (Table 2 and FIGS. 3A, 3B). Specifically, the OCR was significantly lower for the thioamide analogues compared with the amide analogues (mean 24% vs. 55%, p<0.001). For the amide group, we found chloride salts were associated with less dark toxicity (higher % expected OCR) than the PF₆⁻salts (mean 66% vs. 38%, p=0.04). Of the chloride salts, photosensitizers 2-Se—Cl, 4-S—Cl, and 8-S—Cl were not associated with any significant dark toxicity at the 5.0×10⁻⁷ M concentration. These results demonstrate that photosensitizers that are rapidly extruded from resting cells are associated with a low potential for bioenergetic impedance and dark toxicity.

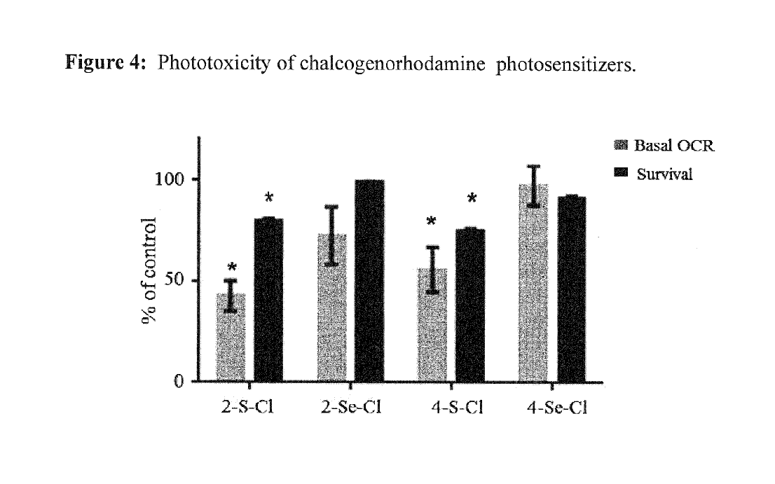

Selective phototoxicity of Chalcogenorhodamine Photosensitizers. Based on the rapid extrusion kinetics and low potential for toxicity in resting cells, we selected four amide-containing half-julolidine analogues for further analysis (2-S—Cl, 2-Se—Cl, 4-S—Cl, and 4-Se—Cl). These photosensitizers are associated with high singlet oxygen quantum yields (Table I). As a result, very low concentrations of these agents are required for phototherapy. For all PD experiments, immunomagnetically-selected CD3⁺ cells were suspended in a photosensitizer-rich media of 5.0×10⁻⁸ M for 20 minutes followed by 30 minutes in a photosensitizer-free media. Cells were then exposed to 5 J/cm² of light followed by real-time measurement of OCR and ECAR. Of the four photosensitizers, only the two selenorhodamine analogues (2-Se—Cl and 4-Se—Cl) did not significantly impede the basal OCR (FIG. 4). We next evaluated the effects of PD on cell survival. For these experiments, FACS analysis was performed 18 hours after PD. Cell survival was identified by failure to bind Annexin 5 and 7AAD, and percent survival was calculated as the difference in the absolute number of cells between PD and control (non-PD samples) samples. Significant cell death occurred with use of the two thiorhodamines analogues (2-S—Cl and 4-S—Cl). In contrast, minimal cell death was observed when the selenorhodamine analogues were used for PD. These results demonstrate that photosensitizers that stimulate P-gp ATPase are rapidly extruded from cells, and protect resting cells from both dark and phototoxicity.

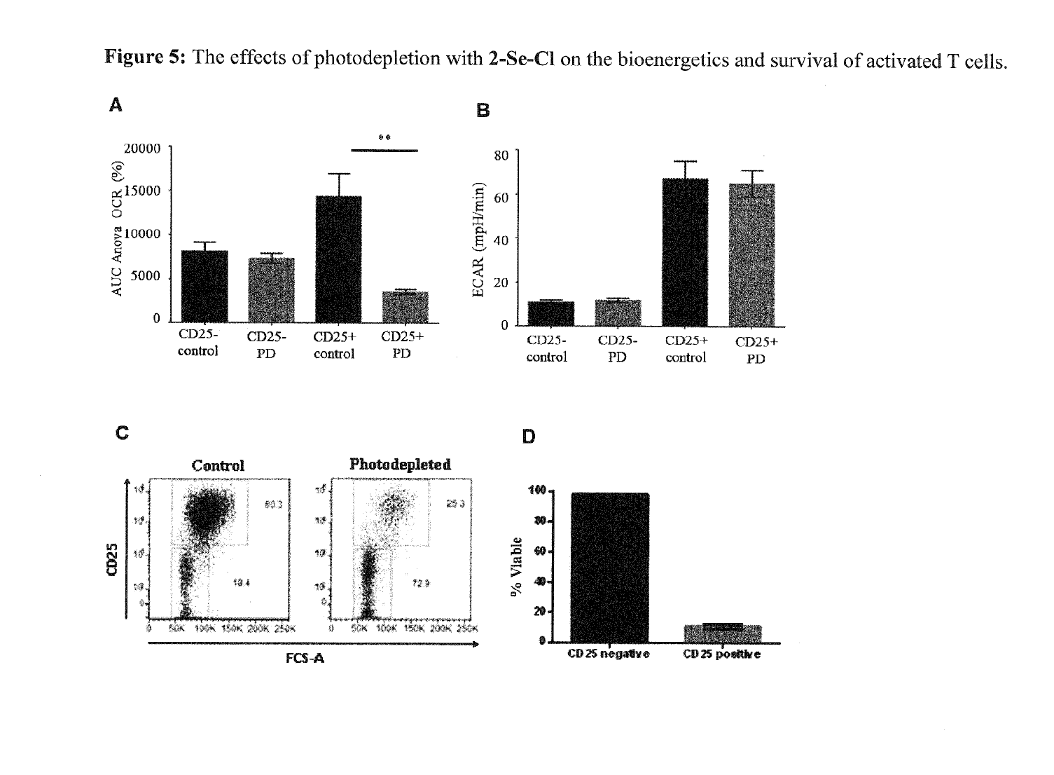

To evaluate the differential effects of PD on bioenergetics of activated and resting T cells, immunomagnetically-selected CD25⁺ and CD25⁻ T cells were isolated (>95% purity) after SEB stimulation. PD was then performed, and bioenergetics were measured within 1 hour. The percent of basal OCR devoted to ATP production was determined by comparing basal OCR to baseline OCR (after oligomycin injection). PD with 2-Se—Cl significantly impeded oxidative phosphorylation (OXPHOS) associated ATP production in activated T cells, but not of resting T cells from the same culture (FIG. 5A), and while not affecting aerobic glycolysis of either population (FIG. 5B). These results indicate that the increased mitochondrial metabolism drives the potential for greater photosensitizer accumulation. Upon exposure to light, the higher concentration of 2-Se—Cl selectively disrupted OXPHOS in activated T cells. To determine whether the selective impedance of basal ATP production affected cell survival, we performed FACS analysis 18 hours after PD (FIG. 5C). Greater than 90% of activated T cells were eliminated from culture with minimal to no cell death occurring in the resting T cell population (FIG. 5D). These results demonstrate that PD with 2-Se—Cl selectively disrupts OXPHOS in activated T cells to induce cell death, while resting T cells remain intact.

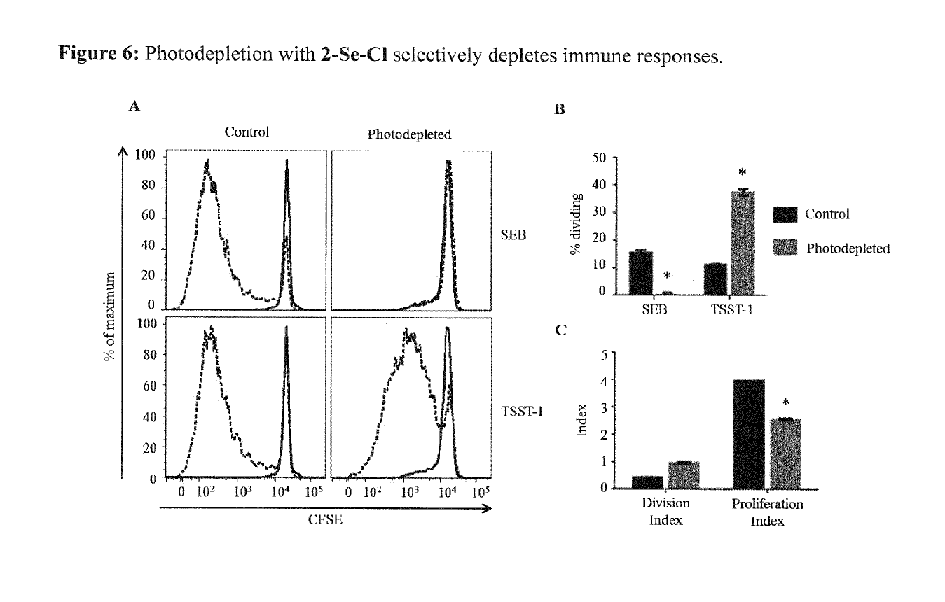

PD with 2-Se—Cl selectively depletes immune responses. PBMCs were stimulated with 50 ng/mL staphylococcal enterotoxin B (SEB) for 72 hours, and then photodepleted using 2-Se—Cl as described above. Cells were then rested overnight, stained with CFSE, and rechallenged with SEB or toxic shock syndrome toxin 1 (TSST-1) in culture for 6 days. After PD, no proliferation occurred in response to SEB (FIG. 6A right upper panel). In contrast, when challenged with TSST-1, a superantigen that stimulates a different range of the T cell receptor (TCR) repertoire compared to SEB, a robust response was observed (FIG. 6A right lower panel). Both SEB and TSST-1 bind to specific TCR sequences, which represent about 20% of the TCR repertoire. The loss of SEB-specific T cells enriched the TSST-1-specific T cells in the remaining PBMCs, and accounts for the increase percentage of dividing cells and the higher division index (the average # of cell divisions for all cells) in response to TSST-1 (FIGS. 7B and C). These studies demonstrate that chalcogenorhodamine photosensitizers designed to modulate P-gp will selectively accumulate in activated T cells to inhibit OXPHOS, and as a result, will selectively deplete an immune response while leaving intact resting cells with a normal response potential.

Discussion. Two approaches to ECP have involved non-selective photosensitizers and poor clinical outcomes. DNA cross-linking by 8-MOP is indiscriminate and occurs in all cells including non-malignant and resting lymphocytes.^{4,5} The use of dibromorhodamine-123 has also resulted in the non-selective depletion of lymphocytes important for normal immune responses, and poor patient outcomes.⁷ Reinfusion of these non-targeted cells in the apoptotic state may reduce the presentation of disease specific antigens, or may induce tolerance to prominent lymphocyte antigens.

The rhodamines have long been known to target the mitochondria of transformed cells.²⁵ Bromination of the rhodamine 123 core increases the quantum yield for singlet oxygen generation by the brominated photosensitizer, but it is not clear that the brominated rhodamines retain their mitochondrial specificity.²⁶ Replacing the oxygen atom of the xanthylium core of the rhodamine with a selenium atom produces the selenoxanthylium core and selenorosamines/rhodamines based on this core have been shown to target mitochondria through light fluence-dependent inhibition of cytochrome c oxidase activity in whole cells.¹⁶

Having a mitochondrial-specific photosensitizer should allow increased uptake of photosensitizer in activated or malignant T-cells relative to resting cells and other lymphocytes. However, any appreciable concentration of photosensitizer in resting T-cells and other lymphocytes may lead to apoptosis of these cells during ECP. The scaffolds 1-8 offer a second means for achieving selectivity—selective depletion of the photosensitizer from resting T-cells. The thioamide-containing scaffolds 1-S—PF₆, 3-S—PF₆, 5-S—PF₆ and 7-S—PF₆ inhibit ATPase activity in P-gp while the amide-containing scaffolds 2-S—PF₆, 4-S—PF₆, 6-S—PF₆ and 8-S—PF₆ stimulate ATPase activity.¹⁵ These differences in ATPase activity manifest themselves in the rate of transmembrane movement of the photosensitizer in the secretory direction (P_{BA}, basolateral to apical) and in the ratio of the % cell-associated photosensitizer in thiorhodamine-treated and fully inhibited systems. These data, taken from reference 15, are summarized in Table 4. Values of P_{BA} are 3.5- to 7-fold faster for the amide relative to the corresponding thioamide and the % cell-assocated photosensitizer is 2.5- to 3-fold greater in the thioamides relative to the amides.¹⁵ Increased mitochondrial activity in activated T cells may slow extrusion of the amide analogues from mitochondria and give higher selectivity for activated T cells with minimal dark toxicity and phototoxicity toward resting T cells.

Of the amides examined in this study, the piperidyl 2-thienyl-5-carboxamide derivative 2-Se—Cl may be the leading candidate for subsequent study. This photosensitizer has λₘₐₓ of 618 nm with an associated s of 7.4×10⁴ M⁻¹ cm⁻¹ and produces singlet oxygen with Φ(¹O₂) of 0.48±0.03 (Table 1). Dark toxicity studies showed minimal toxicity with no statistically significant difference in the % expected OCR compared to the OCR of control (photosensitizer free) cells. The photosensitizer 2-Se—Cl, which is actively extruded from resting T-cells, selectively impedes OXPHOS and induces apoptosis in activated T cells at a concentration of 5.0×10⁻⁸ M and irradiation with 5 J cm⁻² of light, resulting in the selective depletion of the activated T cell population and the associated immune response while leaving intact resting cells with a normal response potential.

TABLE 4
Transport and cell association studies of thiorhodamine amide and
thioamide analogues 1-S—PF₆-8-S—PF₆ with MDCK-MDR1 cells.^{a}

| | | Ratio (+/−inhibitor) |
|---|---|---|
| | P_{BA}, | % Cell Associated |
| Compd | nm s⁻¹ | Photosensitizer |
| 1-S—PF₆ | 34 | 1.8 |
| 2-S—PF₆ | 230 | 5.2 |
| 3-S—PF₆ | 69 | 1.2 |
| 4-S—PF₆ | 370 | 3.3 |
| 5-S—PF₆ | 83 | 2.1 |
| 6-S—PF₆ | 220 | 6.1 |
| 7-S—PF₆ | 65 | 1.8 |
| 8-S—PF₆ | 210 | 4.8 |

^{a}Data from reference 15.

### Compound Preparation.

Preparation of N-methyl-N-(1,4,4-trimethyl-6-(5-(piperidine-1-carbonothioyl)thiophen-2-yl)-3,4-dihydro-1H-selenochromeno[3,2-g]quinolin-9(2H)-ylidene)methanaminium hexafluorophosphate (15). n-Butyllithium (1.38 M in hexanes, 2.12 mL, 2.93 mmol) was added dropwise to a solution of N,N-diisopropylamine (0.490 mL, 3.53 mmol) in THF (10 mL) at −78° C. The resulting mixture was stirred 0.5 h and was then transferred via cannula to a solution of piperidin-1-yl(thiophen-2-yl)methanethione (13, 635 mg, 3.00 mmol) in THF (60 mL) at −78° C. The resulting solution was stirred 10 min and then added via cannula to a solution of 9-(dimethylamino)-1,4,4-trimethyl-3,4-dihydro-1H-selenochromeno[3,2-g]quinolin-6(2H)-one (11, 300 mg, 0.751 mmol, 1.00 eq) in THF (30 mL) at ambient temperature. The reaction mixture was heated to 40° C. for 15 min and then cooled to ambient temperature. Glacial acetic acid (2 mL) was added and the reaction mixture was poured into a 10% v/v aqueous HPF₆ solution (300 mL) and stirred 16 h. The mixture was extracted with dichloromethane (3×50 mL). The combined organic extracts were washed with water (50 mL) and concentrated in vacuo. The crude product was recrystallized from ether/CH₂Cl₂ to give 448 mg (80.7%) of 15 as a blue solid, melting point 233-236° C.: ¹H NMR (500 MHz, CD₂Cl₂) δ 7.82 (d, 1H, J=9.5 Hz), 7.56 (s, 1H), 7.23 (d, 1H, J=2.0 Hz), 7.22-7.17 (m, 2H), 7.06 (d, 1H, J=3.5 Hz), 6.93 (dd, 1H, J=9.5, 2.0 Hz), 4.30 (broad s, 2H), 3.99 (broad s, 2H), 3.60 (t, 2H, J=6.0 Hz), 3.27 (s, 3H), 3.25 (s, 6H), 1.79 (t, 8H, J=6.0 Hz), 1.16 (s, 6H); ¹³C NMR (300 MHz, CDCl₃) δ 188.5, 162.2, 152.7, 151.3, 150.4, 148.1, 145.1, 145.0, 144.6, 140.6, 139.6, 139.4, 137.6, 137.4, 135.1, 131.8, 129.9, 129.7, 125.1, 120.7, 120.1, 115.0, 108.7, 108.3, 48.6, 40.5, 40.2, 34.3, 31.9, 28.5, 26.2, 24.5, 24.1, with splitting due to isomerization; HRMS (ESI) m/z 594.1505 (calcd for C₃₁H₃₇N₃S₂⁸⁰Se⁺: 594.1510); λₘₐₓ (CH₂Cl₂) 607 nm (ε 1.18×10⁵ M⁻¹ cm⁻¹), λₘₐₓ (CH₃OH) 617 nm (ε 1.16×10⁵ M⁻¹ cm⁻¹).

Preparation of N-methyl-N-(1,4,4-trimethyl-6-(5-(piperidine-1-carbonyl)thiophen-2-yl)-3,4-dihydro-1H-selenochromeno[3,2-g]quinolin-9(2H)-ylidene)methanaminium hexafluorophosphate (16). Trifluoroacetic anhydride (0.377 mL, 2.71 mmol, 10.0 eq) was added dropwise to a solution of 15 (200 mg, 0.271 mmol) in dichloromethane (30 mL) The reaction mixture was heated at reflux for 12 h and was then cooled to ambient temperature. A solution of 10% sodium carbonate (10 mL) was added. The resulting mixture was extracted with dichloromethane (3×50 mL) and the combined organic extracts were concentrated. The crude product was purified via chromatography on SiO₂ eluted first with 1:9 ether/CH₂Cl₂ and then with MeOH and 1% HPF₆. The product fractions were dissolved in CH₂Cl₂ and the CH₂Cl₂ solution was washed with water (50 mL) and concentrated. The crude product was recrystallized from ether/CH₂Cl₂ to 22.2 mg (11.3%) of 16 as a blue solid, melting point 194-197° C.: ¹H NMR (500 MHz, CD₂Cl₂) δ 7.72 (d, 1H, J=10.0 Hz), 7.52 (s, 1H), 7.41 (d, 1H, J=3.5 Hz), 7.35-7.24 (m, 2H), 7.13 (d, 1H, J=3.5 Hz), 6.89 (d, 1H, J=9.0 Hz), 3.72 (t, 4H, J=5.0 Hz), 3.60 (t, 2H, J=5.0 Hz), 3.29 (s, 3H), 3.25 (s, 6H), 1.82-1.72 (m, 4H), 1.71-1.64 (m, 4H), 1.14 (s, 6H); ¹³C NMR (300 MHz, CDCl₃) δ 162.1, 152.5, 151.1, 150.2, 145.1, 144.7, 140.4, 139.4, 137.3, 135.0, 131.6, 129.8, 128.2, 120.7, 120.0, 114.8, 108.7, 108.3, 48.5, 40.6, 40.2, 34.2, 31.8, 28.5, 26.1, 24.5; HRMS (ESI) m/z 578.1729 (calcd for C₃₁H₃₇N₃OS⁸⁰Se⁺: 578.1739); λₘₐₓ (CH₂Cl₂) 617 nm (ε 9.29×10⁴ M⁻¹ cm⁻¹), λₘₐₓ (CH₃OH) 618 nm (ε 7.44×10⁴ M⁻¹cm⁻¹).

Preparation of N-(6-(5-(diethylcarbamothioyl)thiophen-2-yl)-1,4,4-trimethyl-3,4-dihydro-1H-selenochromeno[3,2-g]quinolin-9(2H)-ylidene)-N-methylmethanaminium hexafluorophosphate (19). n-Butyllithium (1.38 M in hexanes, 0.708 mL, 0.976 mmol) was added dropwise to a solution of N,N-diisopropylamine (0.166 mL, 1.18 mmol) in THF (5 mL) at −78° C. The resulting mixture was stirred 0.5 h before being transferred via cannula to a solution of N,N-diethylthiophene-2-carbothioamide (17, 200 mg, 1.00 mmol) at −78° C. The resulting solution was stirred 0.5 h and then added via cannula to a solution of 11 (100 mg, 0.250 mmol, 1.00 eq) in THF (8 mL) at ambient temperature. The reaction mixture was heated at 40° C. for 15 min and then cooled to ambient temperature. Glacial acetic acid (2 mL) was added and the reaction mixture was poured into a 10% v/v aqueous HPF₆ solution (200 mL) and stirred 16 h. The mixture was extracted with CH₂Cl₂ (3×50 mL) and the organic extracts were combined, dried over MgSO₄, and concentrated. The crude product was purified via column chromatography (SiO₂, 6% MeOH/CH₂Cl₂). The product fractions were collected, concentrated, and then stirred for 1 h with aqueous 1 M KPF₆ in aqueous MeOH. The reaction mixture was extracted with CH₂Cl₂ (3×50 mL) and the combined organic extracts were dried over MgSO₄ and concentrated. The product was recrystallized from ether/CH₂Cl₂ to give 62.1 mg (34%) of 19 as a blue solid, melting point 226-229° C.: ¹H NMR (500 MHz, CD₂Cl₂) δ 7.80 (d, 1H, J=10.0 Hz), 7.59 (s, 1H), 7.26-7.20 (m, 2H), 7.19 (s, 1H), 7.05 (d, 1H, J=4.0 Hz), 6.93 (dd, 1H, J=10.0, 2.0 Hz), 4.12 (br s, 2H), 3.86 (br s, 2H), 3.60 (t, 2H, J=6.0 Hz), 3.27 (s, 3H), 3.25 (s, 6H), 1.79 (t, 2H, J=6.0 Hz), 1.39 (t, 6H, J=6.5 Hz), 1.67 (s, 6H); ¹³C NMR (500 MHz, CD₂Cl₂) δ 189.0, 153.1, 152.2, 150.8, 148.9, 145.1, 144.7, 139.6, 137.9, 135.4, 132.4, 130.0, 124.6, 121.2, 120.6, 115.2, 109.0, 108.4, 49.1, 40.9, 40.4, 34.6, 32.3, 28.6; HRMS (ESI) m/z 582.1531 (calcd for C₃₀H₃₆N₃S₂⁸⁰Se⁺: 582.1510); λₘₐₓ (CH₂Cl₂) 608 nm (ε 1.19×10⁵ M^{-l}cm⁻¹), λₘₐₓ (CH₃OH) 608 nm (ε 8.63×10⁴ M⁻¹ cm⁻¹).

Preparation of N-(6-(5-(diethylcarbamoyl)thiophen-2-yl)-1,4,4-trimethyl-3,4-dihydro-1H-selenochromeno[3,2-g]quinolin-9(2H)-ylidene)-N-methylmethanaminium hexafluorophosphate (20). Trifluoroacetic anhydride (0.308 mL, 2.22 mmol) was added dropwise to a solution of 19 (200 mg, 0.271 mmol) in CH₂Cl₂ (30 mL). The reaction mixture was heated at reflux for 30 h and was then cooled to ambient temperature. A solution of 10% sodium carbonate (10 mL) was added. The resulting mixture was extracted with dichloromethane (3×50 mL) and the combined organic extracts were concentrated. The crude product was purified via chromatography on SiO₂ eluted first with 1:9 ether/CH₂Cl₂ and then with MeOH and 1% HPF₆. The product fractions were dissolved in CH₂Cl₂ and the CH₂Cl₂ solution was washed with water (50 mL) and concentrated. The crude product was recrystallized from ether/CH₂Cl₂ to 68.6 mg (44%) of 20 as a blue solid: ¹H NMR (500 MHz, CD₃CN) δ 7.63 (d, 1H, J=9.5 Hz), 7.52-7.46 (m, 2H), 7.38 (d, 1H, J=2.5 Hz), 7.35 (s, 1H), 7.17 (d, 1H, J=3.5 Hz), 6.96 (dd, 1H, J=9.5, 2.5 Hz), 3.56 (t, 6H, J=6.0 Hz), 3.21 (s, 3H), 3.19 (s, 6H), 1.74 (t, 2H, J=6.0 Hz), 1.25 (t, 6H, J=7.0 Hz), 1.10 (s, 6H); ¹³C NMR (300 MHz, CDCl₃) δ 162.5, 152.4, 151.1, 145.2, 144.7, 141.3, 139.7, 137.2, 135.0, 131.6, 129.9, 127.9, 120.7, 120.0, 114.7, 108.8, 108.4, 48.5, 40.6, 40.3, 34.2, 31.8, 28.5; HRMS (ESI) m/z 566.1745 (calcd for C₃₀H₃₆N₃OS⁸⁰Se⁺: 566.1739); λₘₐₓ (CH₂Cl₂) 607 nm (ε 1.28×10⁵ M⁻¹ cm⁻¹), λₘₐₓ (CH₃OH) 617 nm (ε 1.04×10⁵ M⁻¹cm⁻¹).

12-(Dimethylamino)-2,3,6,7-tetrahydro-9-(N-piperidyl-2-thienyl-5-carboxamido)-1H,5H-selenoxantheno[2,3,4-ij]quinolizin-14-ium hexafluorophosphate (23). Trifluoroacetic anhydride (189 μL, 1.36 mmol) and 15 (200 mg, 0.271 mmol) in CH₂Cl₂ (30 mL) were treated as described for the preparation of 16. The crude product was purified via column chromatography (SiO₂, 2:8 Et₂O:CH₂Cl₂, R_{f}=0.4), yielding 105 mg (54%) of 23 as a purple solid, m.p. 166-168° C. ¹H NMR (500 MHz, CD₂Cl₂) δ 7.58 (d, 1H, J=9.5 Hz), 7.41 (d, 1H, J=4.0 Hz), 7.34 (s, 1H), 7.25 (d, 1H, J=3.0 Hz), 7.10 (d, 1H, J=4.0 Hz), 6.89 (d×d, 1H, J=3.0, 9.5 Hz), 3.74 (br s, 4H), 3.57-3.51 (m, 4H), 3.22 (s, 6H), 2.80-2.68 (m, 4H), 2.23-2.14 (m, 2H), 2.04-1.94 (m, 2H) 1.79-1.59 (m, 6H); ¹³C NMR (75.5 MHz, CD₂Cl₂) δ 162.2, 152.6, 150.9, 149.6, 143.3, 142.4, 141.2, 140.0, 137.1, 135.1, 130.2, 128.4, 125.9, 121.0, 120.0, 117.2, 114.8, 108.7, 52.0, 51.0, 40.6, 28.0, 26.5 (br), 26.2, 24.8, 20.6, 20.3; λₘₐₓ in CH₂Cl₂ (log ε, M⁻¹ cm⁻¹) 616 nm (5.11); λₘₐₓ in CH₃OH (log ε, M⁻¹ cm⁻¹) 616 nm (5.04); IR (film on NaCl) νₘₐₓ 2934, 1592, 1441 cm⁻¹; HRMS (ESI, HRDFMagSec) m/z 576.1583 (calcd for C₃₀H₃₄N₃O₁S₁⁸⁰Se₁⁺: 576.1582).

For 12-(Dimethylamino)-2,3,6,7-tetrahydro-9-(5-(diethylcarbamothioyl)-thiophen-2-yl)-1H,5H-selenoxantheno[2,3,4-ij]quinolizin-14-ium hexafluorophosphate (24): n-Butyllithium (1.46 M in hexanes, 2.01 mL, 2.94 mmol, 3.9 eq), N,N-diisopropylamine (459 μL, 3.25 mmol, 4.3 eq), N,N-diethylthiophene-2-carbothioamide (61) (602 mg, 3.02 mmol, 4.0 eq), and selenoxanthone 21 (300 mg, 0.755 mmol, 1.0 eq) in THF (10+60+30 mL) were treated as described for the preparation of 19. The product was purified via column chromatography (SiO₂, 1:9 Et₂O:CH₂Cl₂, R_{f}=0.4), followed by recrystallization from CH₂Cl₂/Et₂O, yielding 457 mg (83.5%) of 24 as a purple solid, mp 155-157° C. ¹H NMR (500 MHz, CD₂Cl₂) δ 7.66 (d, 1H, J=9.5 Hz), 7.40 (s, 1H), 7.24 (d, 1H, J=3.0 Hz), 7.23 (d, 1H, J=4.0 Hz), 7.04 (d, 1H, J=4.0 Hz), 6.92 (d×d, 1H, J=2.5, 9.5 Hz), 4.11 (br s, 2H), 3.89 (br s, 2H), 3.57-3.51 (m, 4H), 3.23 (s, 6H), 2.80-2.72 (m, 4H) 2.22-2.16 (m, 2H), 2.04-1.97 (m, 2H), 1.42 (t, 6H, J=7.5 Hz); ¹³C NMR (75.5 MHz, CD₂Cl₂) δ 188.6, 152.5, 150.7, 149.6, 148.8, 143.3, 142.3, 140.6, 137.1, 135.1, 130.1, 125.8, 124.6, 120.9, 119.9, 117.2, 114.9, 108.7, 52.0, 51.0, 48.8 (br), 48.3 (br), 40.6, 28.0, 26.2, 20.6, 20.3, 14.2 (br), 11.2 (br); λₘₐₓ in CH₂Cl₂ (log ε, M⁻¹ cm⁻¹) 616 nm (5.08); λₘₐₓ in CH₃OH (log ε, M⁻¹ cm⁻¹) 615 nm (4.97); IR (film on NaCl) νₘₐₓ 2934, 1591, 1442 cm⁻¹; HRMS (ESI, HRDFMagSec) m/z 580.1358 (calcd for C₃₀H₃₄N₃S₂⁸⁰Se₁⁺: 580.1354).

12-(Dimethylamino)-2,3,6,7-tetrahydro-9-(5-(diethylcarbamoyl)-thiophen-2-yl)-1H,5H-selenoxantheno[2,3,4-ij]quinolizin-14-ium hexafluorophosphate (25). Trifluoroacetic anhydride (231 μL, 1.66 mmol, 5.0 eq) and 24 (240 mg, 0.331 mmol, 1.0 eq) in CH₂Cl₂ (30 mL) were treated as described for the preparation of 16. The resulting product was purified via column chromatography (SiO₂, 2:8 Et₂O:CH₂Cl₂, R_{f}=0.4), yielding 130 mg (55%) of 25 as a purple solid, m.p. 141-143° C. ¹H NMR (500 MHz, CD₂Cl₂) δ 7.59 (d, 1H, J=10.0 Hz), 7.46 (d, 1H, J=3.5 Hz), 7.35 (s, 1H), 7.25 (d, 1H, J=2.5 Hz), 7.11 (d, 1H, J=3.5 Hz), 6.88 (d×d, 1H, J=2.5, 10.0 Hz), 3.62 (br s, 4H), 3.56-3.51 (m, 4H), 3.23 (s, 6H), 2.80-2.69 (m, 4H), 2.23-2.14 (m, 2H), 2.04-1.96 (m, 2H), 1.31 (br s, 6H); ¹³C NMR (75.5 MHz, CD₂Cl₂) δ 162.6, 152.5, 150.8, 149.6, 143.3, 142.3, 142.0, 140.3, 137.1, 135.1, 130.4, 127.9, 125.9, 120.9, 119.9, 117.2, 114.8, 108.7, 51.9, 51.0, 42.6 (br), 40.6, 27.9, 26.2, 20.6, 20.3, 14.2 (br); λₘₐₓ in CH₂Cl₂ (log ε) 615 nm (5.07); in CH₃OH (log c) 616 nm (5.03); IR (film on NaCl) νₘₐₓ 2932, 1592, 1442 cm⁻¹; HRMS (ESI, HRDFMagSec) m/z 564.1594 (calcd for C₃₀H₃₄N₃O₁S₁⁸⁰Se₁⁺: 564.1582).

General Procedure for the Conversion of Hexafluorophosphate Salts to Chloride Salts. Preparation of N-methyl-N-(1,4,4-trimethyl-6-(5-(piperidine-1-carbonothioyl)thiophen-2-yl)-3,4-dihydro-1H-selenochromeno[3,2-g]quinolin-9(2H)-ylidene)methanaminium Chloride (1-Se—Cl). Selenorhodamine 15 (0.212 g, 0.30 mmol) was dissolved in CH₂Cl₂ (15 mL) and Amberlite IRA-400 chloride ion exchange resin (1.0 g) was added and the resulting mixture was stirred for 3 h. The Amberlite ion exchange resin was removed by filtration and the reaction mixture was concentrated. The ion exchange was repeated two additional times to achieve complete exchange of chloride for PF₆⁻. This was repeated three times. The final product was recrystallized from ether/CH₂Cl₂ to give 0.165 g (90%) of 1-Se—Cl as a blue solid, mp 133-236° C.: ¹H NMR (500 MHz, CD₂Cl₂) δ 7.82 (d, 1H, J=9.5 Hz), 7.56 (s, 1H), 7.23 (d, 1H, J=2.0 Hz), 7.22-7.17 (m, 2H), 7.06 (d, 1H, J=3.5 Hz), 6.93 (dd, 1H, J=9.5, 2.0 Hz), 4.30 (broad s, 2H), 3.99 (broad s, 2H), 3.60 (t, 2H, J=6.0 Hz), 3.27 (s, 3H), 3.25 (s, 6H), 1.79 (t, 8H, J=6.0 Hz), 1.16 (s, 6H); ¹³C NMR (300 MHz, CDCl₃) δ 188.5, 162.2, 152.7, 151.3, 150.4, 148.1, 145.1, 145.0, 144.6, 140.6, 139.6, 139.4, 137.6, 137.4, 135.1, 131.8, 129.9, 129.7, 125.1, 120.7, 120.1, 115.0, 108.7, 108.3, 48.6, 40.5, 40.2, 34.3, 31.9, 28.5, 26.2, 24.5, 24.1, with splitting due to isomerization; IR (film on NaCl) 2936, 2360, 1592, 1508, 1474, 1445, 1407, 1386, 1328, 1254, 1213 cm⁻¹; λₘₐₓ (MeOH) 608 nm (ε=1.16×10⁵ M^{-l}cm⁻¹); HRMS (ESI, HRDFMagSec) m/z 594.1505 (calcd for C₃₁H₃₆N₃S₂⁸⁰Se⁺: 594.1510). Anal. Calcd for C₃₁H₃₆ClN₃S₂Se: C, 59.18; H, 5.77; N, 6.68. Found: C, 59.18; H, 5.77; N, 6.68.

For N-methyl-N-(1,4,4-trimethyl-6-(5-(piperidine-1-carbonyl)thiophen-2-yl)-3,4-dihydro-1H-selenochromeno[3,2-g]quinolin-9(2H)-ylidene)methanaminium Chloride (2-Se—Cl). From 16. 192 mg (98%) as a blue solid, mp 194-197° C.: ¹H NMR (500 MHz, CD₂Cl₂) δ 7.72 (d, 1H, J=10.0 Hz), 7.52 (s, 1H), 7.41 (d, 1H, J=3.5 Hz), 7.35-7.24 (m, 2H), 7.13 (d, 1H, J=3.5 Hz), 6.89 (d, 1H, J=9.0 Hz), 3.72 (t, 4H, J=5.0 Hz), 3.60 (t, 2H, J=5.0 Hz), 3.29 (s, 3H), 3.25 (s, 6H), 1.82-1.72 (m, 4H), 1.71-1.64 (m, 4H), 1.14 (s, 6H); ¹³C NMR (300 MHz, CDCl₃) δ 162.1, 152.5, 151.1, 150.2, 145,1, 144.7, 140.4, 139.4, 137.3, 135.0, 131.6, 129.8, 128.2, 120.7, 120.0, 114.8, 108.7, 108.3, 48.5, 40.6, 40.2, 34.2, 31.8, 28.5, 26.1, 24.5; IR (film on NaCl) 2936, 2859, 1592, 1536, 1508, 1473, 1446, 1408, 1387, 1329, 1255, 1214 cm⁻¹; λₘₐₓ (MeOH) 609 nm (ε=7.44×10⁴ M⁻¹cm⁻¹); HRMS (ESI, HRDFMagSec) m/z 578.1739 (calcd for C₃₁H₃₆N₃OS⁸⁰Se⁺: 578.1739). Anal. Calcd for C₃₁H₃₆ClN₃OSSe: C, 60.73; H, 5.92; N, 6.85. Found: C, 60.73; H, 5.92; N, 6.85.

For N-(6-(5-(diethylcarbamothioyl)thiophen-2-yl)-1,4,4-trimethyl-3,4-dihydro-1H-seleno-chromeno[3,2-g]quinolin-9(2H)-ylidene)-N-methylmethanaminium Chloride (3-Se—Cl). From 19. 62 mg, (34%) as a blue solid, mp 162-165° C.: ¹H NMR (500 MHz, CD₂Cl₂) δ 7.80 (d, 1H, J=10.0 Hz), 7.59 (s, 1H), 7.26-7.20 (m, 2H), 7.19 (s, 1H), 7.05 (d, 1H, J=4.0 Hz), 6.93 (dd, 1H, J=2.0, 10.0 Hz), 4.12 (br s, 2H), 3.86 (br s, 2H), 3.60 (t, 2H, J=6.0 Hz), 3.27 (s, 3H), 3.25 (s, 6H), 1.79 (t, 2H, J=6.0 Hz), 1.39 (t, 6H, J=6.5 Hz), 1.67 (s, 6H); ¹³C NMR (500 MHz, CD₂Cl₂) δ 189.0, 153.1, 152.2, 150.8, 148.9, 145.1, 144.7, 139.6, 137.9, 135.4, 132.4, 130.0, 124.6, 121.2, 120.6, 115.2, 109.0, 108.4, 49.1, 40.9, 40.4, 34.6, 32.3, 28.6; IR (film on NaCl) 1592, 1506, 1472, 1446, 1407, 1386, 1356, 1329, 1254, 1212 cm⁻¹; λₘₐₓ (MeOH) 608 nm (ε=8.63×10⁴ M⁻¹cm⁻¹); HRMS (ESI, HRDFMagSec) m/z 582.1511 (calcd for C₃₀H₃₆N₃S₂⁸⁰Se⁺: 582.1510). Anal. Calcd for C₃₀H₃₆ClN₃S₂Se: C, 58.38; H, 5.88; N, 6.81. Found: C, 58.38; H, 5.88; N, 6.81.

For N-(6-(5-(diethylcarbamoyl)thiophen-2-yl)-1,4,4-trimethyl-3,4-dihydro-1H-selenochromeno-[3,2-g]quinolin-9(2H)-ylidene)-N-methylmethanaminium Chloride (4-Se—Cl). From 20. 142 mg (44%) as a purple solid, mp 161-164° C.: ¹H NMR (500 MHz, CD₃CN) δ 7.63 (d, 1H, J=9.5 Hz), 7.52-7.46 (m, 2H), 7.38 (d, 1H, J=2.5 Hz), 7.35 (s, 1H), 7.17 (d, 1H, J=3.5 Hz), 6.96 (dd, 1H, J=2.5, 9.5 Hz), 3.56 (t, 6H, J=6.0 Hz), 3.21 (s, 3H), 3.19 (s, 6H), 1.74 (t, 2H, J=6.0 Hz), 1.25 (t, 6H, J=7.0 Hz), 1.10 (s, 6H); ¹³C NMR (300 MHz, CDCl₃) δ 162.5, 152.4, 151.1, 145.2, 144.7, 141.3, 139.7, 137.2, 135.0, 131.6, 129.9, 127.9, 120.7, 120.0, 114.7, 108.8, 108.4, 48.5, 40.6, 40.3, 34.2, 31.8, 28.5; IR (film on NaCl) 1591, 1447, 1386, 1328, 1254 cm⁻¹; λₘₐₓ (MeOH) 609 nm (ε=1.04×10⁵ M^{-l}cm⁻¹); HRMS (ESI, HRDFMagSec) m/z 566.1745 (calcd for C₃₀H₃₆N₃OS⁸⁰Se⁺: 566.1739). Anal. Calcd for C₃₀H₃₆ClN₃OSSe: C, 59.94; H, 6.04; N, 6.99. Found: C, 59.94; H, 6.04; N, 6.99.

12-(Dimethylamino)-2,3,6,7-tetrahydro-9-(N-piperidyl-2-thienyl-5-carboxamido)-1H,5H-selenoxantheno[2,3,4-ij]quinolizin-14-ium hexafluorophosphate(V) (6-Cl—Se). From 23. m.p. 184-186° C. ¹H NMR (500 MHz, CD₂Cl₂) δ 7.58 (d, 1H, J=9.5 Hz), 7.41 (d, 1H, J=4.0 Hz), 7.34 (s, 1H), 7.25 (d, 1H, J=3.0 Hz), 7.10 (d, 1H, J=4.0 Hz), 6.89 (d×d, 1H, J=3.0, 9.5 Hz), 3.74 (br s, 4H), 3.57-3.51 (m, 4H), 3.22 (s, 6H), 2.80-2.68 (m, 4H), 2.23-2.14 (m, 2H), 2.04-1.94 (m, 2H) 1.79-1.59 (m, 6H); ¹³C NMR (75.5 MHz, CD₂Cl₂) δ 162.2, 152.6, 150.9, 149.6, 143.3, 142.4, 141.2, 140.0, 137.1, 135.1, 130.2, 128.4, 125.9, 121.0, 120.0, 117.2, 114.8, 108.7, 52.0, 51.0, 40.6, 28.0, 26.5 (br), 26.2, 24.8, 20.6, 20.3; λₘₐₓ in CH₂Cl₂ (log ε, M⁻¹ cm⁻¹) 616 nm (5.11); λₘₐₓ in CH₃OH (log ε, M⁻¹ cm⁻¹) 616 nm (5.04); IR (film on NaCl) νₘₐₓ 2934, 1592, 1441 cm⁻¹; HRMS (ESI, HRDFMagSec) m/z 576.1583 (calcd for C₃₀H₃₄N₃O₁S₁⁸⁰Se₁⁺: 576.1582).

For 12-(Dimethylamino)-2,3,6,7-tetrahydro-9-(5-(diethylcarbamothioyl)-thiophen-2-yl)-1H,5H-selenoxantheno[2,3,4-ij]quinolizin-14-ium chloriode (7-Cl—Se). From 24. 400 mg (85%), mp 184-186° C. ¹H NMR (500 MHz, CD₂Cl₂) δ 7.66 (d, 1H, J=9.5 Hz), 7.40 (s, 1H), 7.24 (d, 1H, J=3.0 Hz), 7.23 (d, 1H, J=4.0 Hz), 7.04 (d, 1H, J=4.0 Hz), 6.92 (d×d, 1H, J=2.5, 9.5 Hz), 4.11 (br s, 2H), 3.89 (br s, 2H), 3.57-3.51 (m, 4H), 3.23 (s, 6H), 2.80-2.72 (m, 4H) 2.22-2.16 (m, 2H), 2.04-1.97 (m, 2H), 1.42 (t, 6H, J=7.5 Hz); ¹³C NMR (75.5 MHz, CD₂Cl₂) δ 188.6, 152.5, 150.7, 149.6, 148.8, 143.3, 142.3, 140.6, 137.1, 135.1, 130.1, 125.8, 124.6, 120.9, 119.9, 117.2, 114.9, 108.7, 52.0, 51.0, 48.8 (br), 48.3 (br), 40.6, 28.0, 26.2, 20.6, 20.3, 14.2 (br), 11.2 (br); λₘₐₓ in CH₂Cl₂ (log ε, M⁻¹ cm⁻¹) 616 nm (5.08); λₘₐₓ in CH₃OH (log ε, M⁻¹ cm⁻¹) 615 nm (4.97); IR (film on NaCl) νₘₐₓ 2934, 1591, 1442 cm⁻¹; HRMS (ESI, HRDFMagSec) m/z 580.1358 (calcd for C₃₀H₃₄N₃S₂⁸⁰Se₁⁺: 580.1354).

12-(Dimethylamino)-2,3,6,7-tetrahydro-9-(5-(diethylcarbamoyl)-thiophen-2-yl)-1H,5H-selenoxantheno[2,3,4-ij]quinolizin-14-ium Chloride (8-Cl—Se). From 25. 130 mg (55.3%) as a purple solid, m.p. 150-152° C. ¹H NMR (500 MHz, CD₂Cl₂) δ 7.59 (d, 1H, J=10.0 Hz), 7.46 (d, 1H, J=3.5 Hz), 7.35 (s, 1H), 7.25 (d, 1H, J=2.5 Hz), 7.11 (d, 1H, J=3.5 Hz), 6.88 (d×d, 1H, J=2.5, 10.0 Hz), 3.62 (br s, 4H), 3.56-3.51 (m, 4H), 3.23 (s, 6H), 2.80-2.69 (m, 4H), 2.23-2.14 (m, 2H), 2.04-1.96 (m, 2H), 1.31 (br s, 6H); ¹³C NMR (75.5 MHz, CD₂Cl₂) δ 162.6, 152.5, 150.8, 149.6, 143.3, 142.3, 142.0, 140.3, 137.1, 135.1, 130.4, 127.9, 125.9, 120.9, 119.9, 117.2, 114.8, 108.7, 51.9, 51.0, 42.6 (br), 40.6, 27.9, 26.2, 20.6, 20.3, 14.2 (br); λₘₐₓ in CH₂Cl₂ (log ε) 615 nm (5.07); λₘₐₓ in CH₃OH (log ε) 616 nm (5.03); IR (film on NaCl) νₘₐₓ 2932, 1592, 1442 cm⁻¹; HRMS (ESI, HRDFMagSec) m/z 564.1594 (calcd for C₃₀H₃₄N₃O₁S₁⁸⁰Se₁⁺: 564.1582).

Determination of Singlet Oxygen Yields from Singlet Oxygen Phosphorescence Spectroscopy. Generation of singlet oxygen (¹O₂) was assessed by its phosphorescence peaked at 1270 nm. A SPEX 270M spectrometer (Jobin Yvon, Longjumean, France) equipped with IR-PMT photodetector (Hamamatsu, Japan) Electrooptical Systems Inc., Phoenixville, Pa.) was used for acquisition of the emission spectra in NIR spectral range. A diode-pumped solid-state laser (Millenia, Spectra Physics) at 532 nm was the excitation source. The decays of this emission were acquired using the Infinium oscilloscope (Hewlett-Packard, Palo Alto, Calif.) coupled to the output of the excitation source, which is attached to the second output port of the SPEX 270M spectrometer. The emission signal was collected at 90-degrees relative to the exciting laser beam with the use of additional long-pass filters (a 950LP filter and/or a 538AELP filter) to attenuate the scattered light and fluorescence from the samples. The samples (methanol solutions of the compounds in quarts cuvettes) were placed in front of the spectrometer entrance slit. A second harmonic (532 nM) from the nanosecond pulsed Nd:YAG laser (Lotis TII, Belarus) operating at 20 Hz was used as the excitation source for time-resolved measurements.

Fluorescence Quantum Yields (Φ_{F}). All samples were measured in 1-cm² quartz cuvettes. Electronic absorbance measurements were acquired by using a Hewlett Packard diode array spectrometer. Emission spectra were acquired on a SLM AMINCO model 8100 fluorimeter (λₑₓ: 532 nm). A single emission monochromator scanned a range of emission wavelengths which were detected using a photomultiplier tube. A reference channel was used simultaneously with the standard reference fluorophore (TMR-S, Φ_{F}=0.21). Methanol was used as a blank for electronic absorbance and emission measurement. Three samples were prepared for each concentration of TMR-S and chalcogenorhodamines 5-S—PF₆, 5-S—Cl, 5-Se—Cl, 6-S—PF₆, 6-S—Cl, and 6-Se—Cl. Triplicates measurements were recorded for electronic absorption and fluorescence. Relative fluorescence values (R.F.) were determined in 1% BSA and 10% MeOH in pH 7.4 phosphate buffer for samples with an optical density of 0.1 at the excitation wavelength of 532 nm.

### REFERENCES

(1) Worel, N.; Leitner, G. Clinical results of extracorporeal photopheresis. Transfus. Med. Hemother. 2012, 39, 254-262.

(2) Quaglino, P.; Knobler, R.; Fierro, M. T.; et al. Extracorporeal photopheresis for the treatment of erythrodermic cutaneous T-cell lymphoma: a single center clinical experience with long-term follow-up data and a brief overview of the literature. Int. J. Dermatol. 2013.

(3) Girardi, M.; Berger, C. L.; Wilson, L. D.; et al. Transimmunization for cutaneous T cell lymphoma: a Phase I study. Leuk. Lymphoma. 2006, 47, 1495-1503.

(4) Evans, A. V.; Wood, B. P.; Scarisbrick, J. J.; et al. Extracorporeal photopheresis in Sezary syndrome: hematologic parameters as predictors of response. Blood 2001, 98, 1298-1301.

(5) Holtick, U.; Wang, X. N.; Marshall, S. R.; Scheid, C.; von Bergwelt-Baildon, M.; Dickinson, A. M. Immature DC Isolated After Co-Culture with PUVA-Treated Peripheral Blood Mononuclear Cells Downregulate Graft-Versus-Host Reactions in the Human Skin Explant Model. Curr. Stem Cell Res. Ther. 2013, 8, 324-332.

(6) McIver, Z. A.; Melenhorst, J. J.; Grim, A.; et al. Immune reconstitution in recipients of photodepleted HLA-identical sibling donor stem cell transplantations: T cell subset frequencies predict outcome. Biol. Blood Marrow Transplant. 2011, 17, 1846-1854.

(7) Mielke, S; McIver, Z. A.; Shenoy, A.; et al. Selectively T cell-depleted allografts from HLA-matched sibling donors followed by low-dose posttransplantation immunosuppression to improve transplantation outcome in patients with hematologic malignancies. Biol. Blood Marrow Transplant. 2011, 17, 1855-1861.

(8) Gottesman, M. M.; Fojo, T.; Bates, S. E. Multidrug resistance in cancer: role of ATP-dependent transporters. Nat. Rev. Cancer. 2002, 2, 48-58.

(9) Dantzig, A. H.; de Alwis, D. P.; Burgess, M. Considerations in the design and development of transport inhibitors as adjuncts to drug therapy. Adv. Drug Delivery Rev. 2003, 55, 133-150.

(10) Sandor, V.; Fojo, T.; Bates, S. E. Future perspectives for the development of P-glycoprotein modulators. Drug Resistance Updates 1998, 1, 190-200.

(11) Mahon, F. X.; Deininger, M. W. N.; Schultheis, B.; Chabrol, J.; Reiffers, J.; Goldman, J. M.; Melo, J. V. Selection and characterization of BCR-ABL positive cell lines with differential sensitivity to the tyrosine kinase inhibitor STI571: diverse mechanisms of resistance. Blood 2000, 96, 1070-1079.

(12) Tsuruo, T.; Iida, H.; Tsukagoshi, S.; Sakurai, Y. Overcoming of vincristine resistance in P388 leukemia in vivo and in vitro through enhanced cytotoxicity of vincristine and vinblastine by verapamil. Cancer Res. 1981, 41, 1967-1972.

(13) Loo T W, Clarke D M. Do drug substrates enter the common drug-binding pocket of P-glycoprotein through “gates”? Biochem. Biophys. Res. Commun. 2005, 329, 419-422.

(14) Gannon, II, M. K.; Holt, J. J.; Bennett, S. M.; Wetzel, B. R.; Loo, T. W.; Bartlett, M. C.; Clarke, D. M.; Sawada, G. A.; Higgins, J. W.; Tombline, G.; Raub, T. J.; Detty, M. R. Rhodamine inhibitors of P-glycoprotein: An amide/thioamide “switch” for ATPase activity. J. Med. Chem. 2009, 52, 3328-3341.

(15) Orchard, A.; Schamerhorn, G. A.; Calitree, B. D.; Sawada, G. A.; Loo, T. W.; Bartlett, M. C.; Clarke, D. M.; Detty, M. R. Thiorhodamines containing amide and thioamide functionality as inhibitors of the ATP-binding cassette drug transporter P-glycoprotein (ABCB1). Bioorg. Med. Chem. 2012, 20, 4290-4302.

(16) Detty, M. R.; Prasad, P. N.; Donnelly, D. J.; Ohulchanskyy, T.; Gibson, S. L.; Hilf, R. Synthesis, properties, and photodynamic properties In vitro of heavy-chalcogen analogues of tetramethylrosamine. Bioorg. Med. Chem. 2004, 12, 2537-2544

(17) Holt, J. J.; Gannon, M. K.; Tombline, G.; McCarty, T. A.; Page, P. M.; Bright, F. V.; Detty, M. R. A cationic chalcogenoxanthylium photosensitizer effective in vitro in chemosensitive and multidrug-resistant cells. Biorg. Med. Chem. 2006, 14, 8635-8643

(18) Ohulchanskyy, T.; Donnelly, D. J.; Detty, M. R.; Prasad, P. N. Heteroatom substitution changes in excited-state photophysics and singlet oxygen generation in chalcogenoxanthylium dyes: Effect of sulfur and selenium substitutions. J. Phys. Chem. B. 2004, 108, 8668-8672

(19) Del Valle, D. J.; Donnelly, D. J.; Holt, J. J.; Detty, M. R. 2,7-Bis-N,N-dimethylamino-chalcogenoxanthen-9-ones via electrophilic cyclization with phosphorus oxychloride. Organometallics 2005, 24, 3807-3810.

(20) Brennan, N. K.; Donnelly, D. J.; Detty, M. R. Selenoxanthones via directed metallations in 2-arylselenobenzamide derivatives. J. Org. Chem. 2003, 68, 3344.

(21) Zhu, W.; Li, Z.; Zhang, Y. New method for the synthesis of 2-aminoallyl compounds. Hecheng Huaxue 2005, 13, 471-473.

(22) Holt, J. J.; Calitree, B. D.; Vincek, J.; Gannon, M. K., II; Detty, M. R. A microwave-assisted synthesis of julolidine-9-carboxamide derivatives and their conversion to chalcogenoxanthones via directed metalation. J. Org. Chem. 2007, 72, 2690-2693.

(23) Ohulchanskyy, T. Y.; Roy, I.; Goswami, L. N.; Chen, Y.; Bergey, E. J.; Pandey, R. K.; Oseroff, A. R.; Prasad, P. N. Organically modified silica nanoparticles with covalently incorporated photosensitizer for photodynamic therapy of cancer. Nano Lett. 2007, 7, 2835-2842.

(24) Guimond, M.; Balassy, A.; Barrette, M.; Brochu, S.; Perreault, C.; Roy, D. C. P-glycoprotein targeting: a unique strategy to selectively eliminate immunoreactive T cells. Blood. 2002, 100, 375-382.

(25) (a) Bernal, S. D.; Lampdis, T. J.; McIsaac, R. M.; Chen, L. B. Anticarcinoma activity in vivo of rhodamine 123, a mitochondrial-specific dye. Science 1986, 222, 169-172. (b) Davis, S.; Weiss, M. J.; Wong, J. R.; Lampidis, T. J.; Chen, L. B. Mitochondrial and plasma membrane potentials cause unusual accumulation and retention of rhodamine 123 by breast adenocarcinoma-derived MCF-7 cells. J. Biol. Chem. 1985, 260, 13844-13850. (c) Johnson, L. V.; Walsh, M. L.; Bockus, B. J.; Chen, L. B. Monitoring of relative mitochondrial membrane potential in living cells by fluorescence microscopy. J. Cell. Biol. 1981, 88, 526-535.

(26) Ogata, M.; Inanami, O.; Nakajima, M.; Nakajima, T.; Hiraoka, W.; Kuwabara, M. Ca2+-dependent and caspace-3-independent apoptosis caused by damage in Golgi apparatus due to 2,4,5,7-tetrabromorhodamine 123 bromide-induced photodynamic effects. Photochem. Photobiol. 2003, 78, 241-247.

The foregoing is illustrative of the present invention, and not to be construed as limiting thereof. The invention is defined by the following claims, with equivalents of the claims to be included therein.

1. A method of selectively depleting pathogenic T lymphocytes from a blood cell composition, comprising:
(a) combining said cell composition ex vivo with an active compound in an effective amount, and then
(b) irradiating said cells with light ex vivo for a time and at an intensity sufficient to selectively kill pathogenic T lymphocytes in said cell composition;
wherein said active compound is selected from the group consisting of:
(i) compounds of Formula I: 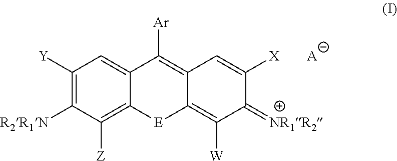
wherein:
E is S or Se;
Ar is aryl or heteroaryl, each of which is substituted or unsubstituted;
W, X, Y, and Z are each independently H or C1 through C8, linear or branched, alkyl;
R₁′, R₂′, R₁″ and R₂″ are each independently H or C1 through C8, linear or branched, alkyl; and/or
R₁′ and R₂′ are alkyl groups connected such that they together comprises a 3, 4, 5, 6 or 7-membered ring, which ring optionally bears alkyl or aryl substituents; and/or
R₁″ and R₂″ are alkyl groups connected such that they together comprises a 3, 4, 5, 6 or 7-membered ring, which ring optionally bears alkyl or aryl substituents; and/or
R₁′ and Y are connected such that they together comprises a 5, 6 or 7-membered ring; and/or
R₁′ and Y are connected such that they together comprises a 5, 6 or 7-membered ring; and/or
R₂′ and Z are connected such that they together comprises a 5, 6 or 7-membered ring; and/or
R₁″ and W are connected such that they together comprises a 5, 6 or 7-membered ring; and/or
R₂″ and X are connected such that they together comprises a 5, 6 or 7-membered ring; and
A is an anion;
(ii) compounds of Formula Ia: 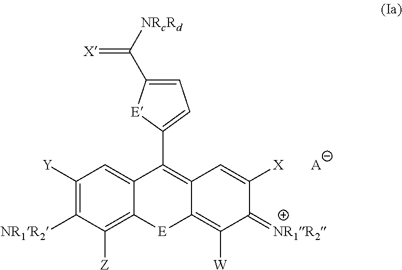
wherein:
E is S or Se;
E′ is O, S, NH, or NRₑ wherein Rₑ is C1 to C6, linear or branched, alkyl;
X′ is O or S;
W, X, Y, and Z, and R₁′, R₂′, R₁″ and R₂″, are as described in connection with Formula I above; and
R_{c} and R_{d} are each independently H or C1 to C6, linear or branched, alkyl, or R_{c} and R_{d} together form with N a 3, 4, 5 6, or 7 membered ring; and
A is an anion;
(iii) compounds of Formula Ib: 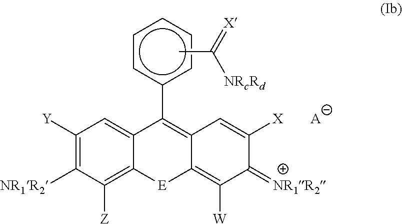
wherein:
E is S or Se;
X′ is O or S;
W, X, Y, and Z, and R₁′, R₂′, R₁″ and R₂″, and R_{c} and R_{d}, are as described in connection with Formula I above;
the group —C(═X′)(—NR_{c}R_{d}) can be in the 2—(ortho), 3—(meta), or 4—(para) position; and
A is an anion;
(iv) compounds of Formula IIa: 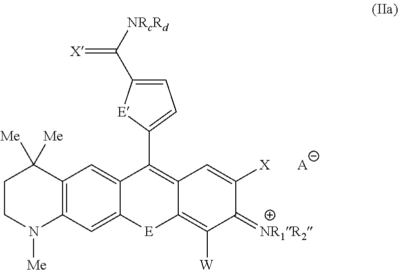
wherein:
E is S or Se;
E′ is O, S, NH, or NRₑ, wherein Rₑ is C1 to C6, linear or branched, alkyl;
X′ is O or S;
W and X, R₁″ and R₂″, and R_{c} and R_{d}, are as described in connection with Formula I above; and
A is an anion;
(v) compounds of Formula IIb: 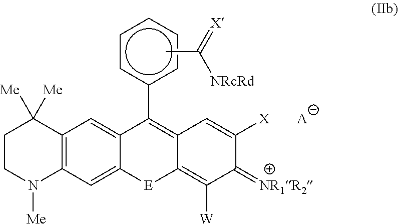
wherein:
E is S or Se;
X′ is O or S;
W and X, R₁″ and R₂″, and R_{c} and R_{d}, are as described in connection with Formula I above;
the group —C(═X′)(—NR_{c}R_{d}) can be in the 2—(ortho), 3—(meta), or 4—(para) position; and
A is an anion;
(vi) compounds of Formula IIIa: 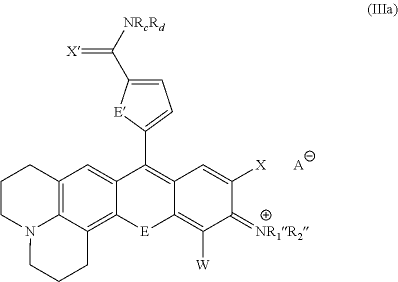
wherein:
E is S or Se;
E′ is O, S, NH, or NRₑ, wherein Rₑ is C1 to C6, linear or branched, alkyl;
X′ is O or S;
W and X, R₁″ and R₂″, and R_{c} and R_{d}, are as described in connection with Formula I above; and
A is an anion;
(vii) compounds of Formula IIIb: 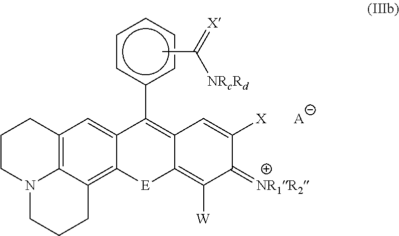
wherein:
E is S or Se;
X′ is O or S;
W and X, R₁″ and R₂″, and R_{c} and R_{d}, are as described in connection with Formula I above;
the group —C(═X′)(—NR_{c}R_{d}) can be in the 2—(ortho), 3—(meta), or 4—(para) position; and
A is an anion;
(viii) compounds of Formula IVa: 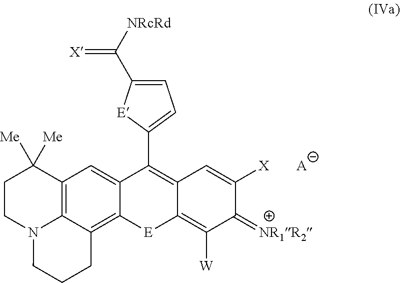
wherein:
E is S or Se;
E′ is O, S, NH, or NRₑ, wherein Rₑ is C1 to C6, linear or branched, alkyl;
X′ is O or S;
W and X, R₁″ and R₂″, and R_{c} and R_{d}, are as described in connection with Formula I above; and
A is an anion;
(ix) compounds of Formula IVb: 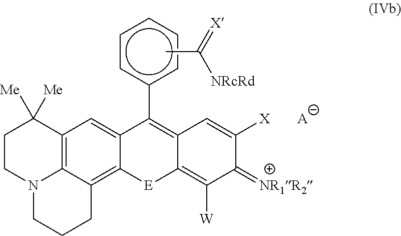
wherein:
E is S or Se;
X′ is O or S;
W and X, R₁″ and R₂″, and R_{c} and R_{d}, are as described in connection with Formula I above;
the group —C(═X′)(—NR_{c}R_{d}) can be in the 2—(ortho), 3—(meta), or 4—(para) position; and
A is an anion;
(x) compounds of Formula Va: 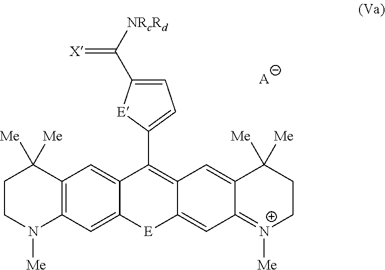
wherein:
E is S or Se;
E′ is O, S, NH, or NRₑ, wherein Rₑ is C1 to C6, linear or branched, alkyl;
X′ is O or S;
R_{c} and R_{d} are as described in connection with Formula I above; and
A is an anion;
(xi) compounds of Formula Vb: 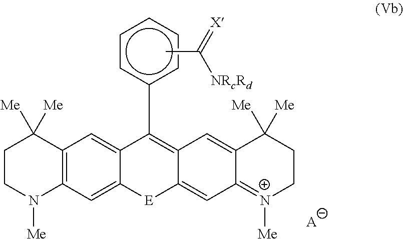
wherein:
E is S or Se;
X′ is O or S;
R_{c} and R_{d} are as described in connection with Formula I above;
the group —C(═X′)(—NR_{c}R_{d}) can be in the 2—(ortho), 3—(meta), or 4—(para) position; and
A is an anion;
(xii) compounds of Formula VIa: 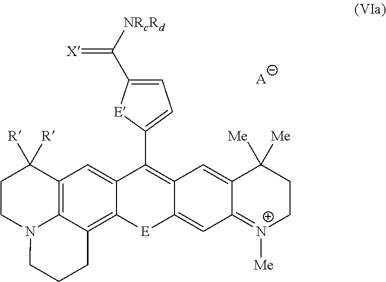
wherein:
E is S or Se;
E′ is O, S, NH, or NRₑ, wherein Rₑ is C1 to C6, linear or branched, alkyl;
X′ is O or S;
R_{c} and R_{d} are as described in connection with Formula I above; and
each R′ is independently H or C1 to C6, linear or branched, alkyl; and
A is an anion;
(xiii) compounds of Formula VIb: 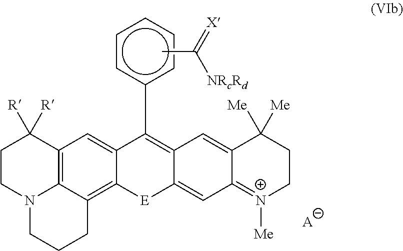
wherein:
E is S or Se;
X′ is O or S;
R_{c} and R_{d} are as described in connection with Formula I above;
each R′ is independently H or alkyl;
the group —C(═X′)(—NR_{c}R_{d}) can be in the 2—(ortho), 3—(meta), or 4—(para) position; and
A is an anion;
(xiv) compounds of Formula VIIa: 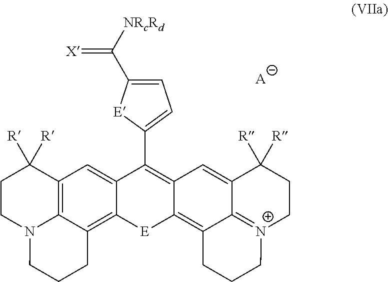
wherein:
E is S or Se;
E′ is O, S, NH, or NRₑ, wherein Rₑ is C1 to C6, linear or branched, alkyl;
X′ is O or S; and
each R′ is independently H or alkyl;
each R″ is independently H or alkyl;
R_{c} and R_{d} are as described in connection with Formula I above; and
A is an anion; and
(xv) compounds of Formula VIIb: 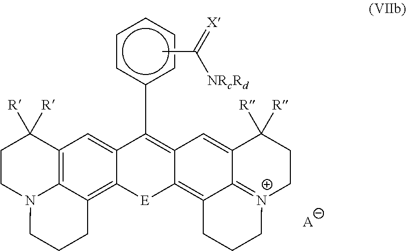
wherein:
E is S or Se;
X′ is O or S;
each R′ is independently H or alkyl;
each R″ is independently H or alkyl;
R_{c} and R_{d} are as described in connection with Formula I above; and
the group —C(═X′)(—NR_{c}R_{d}) can be in the 2—(ortho), 3—(meta), or 4—(para) position; and
A is an anion.

2. The method of claim 1, wherein E is S.

3. The method of claim 1, wherein E is Se.

4. The method of claim 1, wherein E′ when present is S.

5. The method of claim 1, wherein X′ is O.

6. The method of claim 1, wherein A is chloride.

7. The method of claim 1, wherein said pathogenic T lymphocytes are alloreactive T-lymphocytes.

8. The method of claim 1, wherein said pathogenic T lymphocytes are malignant T-lymphocytes.

9. The method of claim 1, wherein said pathogenic T-lymphocytes are autoreactive T-lymphocytes in a blood cell composition collected from a subject afflicted with an autoimmune disease.

10. The method of claim 9, wherein said autoimmune disease is selected from the group consisting of: graft versus host disease (GVHD), solid organ transplant rejection, scleroderma, atopic dermatitis, epidermolysis bullosa acquisita, lichen planus, lupus erythematosus, pemphigus vulgaris, Crohn disease, type 1 diabetes, psoriasis, rheumatoid arthritis, multiple sclerosis, nephrogenic systemic fibrosis/nephrogenic fibrosing dermopathy, and scleromyxedema.

11. The method of claim 1, wherein said blood cell composition comprises a biological fluid.

12. The method of claim 11, wherein said biological fluid is selected from the group consisting of: (i) whole blood, (ii) a white blood cell-containing fraction of whole blood, and (iii) a hematopoietic stem cell-containing fraction of blood or tissue.

13. The method of claim 1, wherein said irradiating step is carried out with an artificial source of ultraviolet light.

14. The method of claim 1, wherein said irradiating step is carried out continuously under sterile conditions in an enclosed fluid circuit containing said blood cell composition.

15. The method of claim 1, further comprising the step of:
(c) administering said cells after said irradiating step to a subject in need thereof.
